(19) United States
(12) Patent Application Publication
(10) Pub. No.: US 2022/0176095 A1
(43) Pub. Date: Jun. 9, 2022

(54) MICRONEEDLE HAVING STRUCTURE OF THREE OR MORE LAYERS, AND METHOD FOR MANUFACTURING SAME

(71) Applicant: Feroka Inc., Seoul (KR)

(72) Inventors: Induk LEE, Seoul (KR); Yeo Myung LIM, Seoul (KR); Yiseul JEON, Seoul (KR); Yeon-wook SUNG, Seoul (KR)

(21) Appl. No.: 17/593,049

(22) PCT Filed: Mar. 6, 2020
(86) PCT No.: PCT/KR2020/003151

§ 371 Date: Sep. 7, 2021

(30) Foreign Application Priority Data
Mar. 8, 2019 (KR) 10-2019-0026946
Mar. 8, 2019 (KR) 10-2019-0026947
Mar. 8, 2019 (KR) 10-2019-0026948

(51) Int. Cl. A61M 37/00

(52) U.S. Cl. CPC A61M 37/0015; A61M 2037/0046; A61M 2037/0023; A61M 2037/0053

(57) ABSTRACT

The present invention pertains to: a microneedle having a structure of three or more layers; and a method for manufacturing same. The microneedle has a structure of three or more layers, wherein the structure is a structure including an inner pillar shell, a structure including a three-dimensional structure shell, or a structure including a solid drug.

### TECHNICAL FIELD

The present invention relates to a microneedle in a structure of three or more layers and a manufacturing method thereof.

### RELATED ART

In the case of injecting a physiologically active material into the skin of a human, an existing injection needle may be used, which may cause pain at an injection site, damage bleeding from the skin, and disease injection by the injection needle.

Therefore, in the recent times, a method for intradermal delivery of a physiologically active material using a microneedle or an ultra-fine needle is actively studied. The microneedle may have a diameter of tens to hundreds of micrometers to penetrate the stratum corneum of the skin that is a main barrier layer.

Dissimilar to the existing injection needle, the microneedle may implement painless skin puncture and no trauma. Also, since the microneedle needs to penetrate the stratum corneum of the skin, a predetermined degree of physical hardness may be required. Also, an appropriate length may be required for the physiologically active material to reach an epidermal layer or a dermal layer of the skin. Also, for the physiologically active material of hundreds of microneedles to be effectively delivered into the skin, the skin permeability of the microneedle needs to be high and needs to be maintained during a predetermined period of time from insertion into the skin to dissolution.

An existing method of manufacturing such a microneedle may include a mold manufacturing method and a tension manufacturing method.

Due to characteristics of a mold, a microneedle manufacturing method using a mold scheme has a low aspect ratio of a microneedle and thus, it is difficult to puncture the skin and a number density of microneedles is low.

A microneedle manufacturing method using a tension scheme refers to a method of manufacturing a microneedle by dropping a material on a patch, stretching and drying the patch, and then truncating a thinned portion. Due to such a characteristic, a length of each microneedle is inconsistent and a lot of pain may occur due to a shape of the microneedle.

Also, both the mold scheme and the tension scheme are expensive, which is an obstacle in terms of a market growth. Since microneedles are not arranged at a high density, there is an inconvenience in that the microneedles need to be attached for about 2 hours. In addition, since the above two schemes have difficulty in increasing the number density of microneedles in terms of construction methods, it is recommended that microneedle patches manufactured using the two methods be attached for 2 hours or more, which is longer than a general patch that is recommended to be attached for about 20 minutes.

Due to the low number density of needles, a long attachment time is required. Since the number density of microneedles is low, the entire surface area of microneedles included in a patch is narrow. Since a contact area with the skin is narrow, a reaction rate with the skin is inevitably low. Here, the existing two schemes have difficulty in increasing the number density and thus, may not further accelerate a reaction rate with the skin.

In the case of manufacturing a microneedle not for cosmetic use but for medical use, the limitations of the existing two schemes are further revealed. When mixing vaccines or drugs, the existing two schemes need to prepare the entire needles using a homogeneous mixture of the same concertation. However, due to a difficulty in making a needle size constant and a drug remaining in an interface between a patch and the skin or in an injection passage, a degree of penetration into the skin may not be adjusted and quantitative administration is almost impossible.

Accordingly, a need for a microneedle in a multilayered structure has been raised. For example, it has been argued that quantitative insulin administration requires such a multilayered microneedle (Ito et al., Diabetes Technology & Therapeutics, 2012, 14, 10).

### DETAILED DESCRIPTION

### Subject

The present invention provides a microneedle that may adjust a melting rate of a lower portion due to an increase in a surface area by a hollow inner pillar shell, may enhance preservation of a drug, and may facilitate penetration into the skin by manufacturing a tree-shaped microneedle in a structure of three or more layers including an upper portion, a middle portion, and a lower portion formed using a single inner pillar shell or a plurality of inner pillar shells.

The present invention provides a microneedle that may adjust a melting rate of a lower portion due to an increase in the surface area by a 3D structure shell, may enhance preservation of a drug, and may facilitate penetration into the skin by manufacturing a tree-shaped microneedle in a structure of three or more layers including an upper portion, a middle portion, and a lower portion formed using the 3D structure shell.

The present invention provides a microneedle that may enhance preservation of a drug and may penetrate, into the skin, a solid drug in a structure in which a drug is contained by manufacturing a tree-shaped microneedle in a structure of three or more layers including a middle portion containing the solid drug in a cavity, a lower portion configured to support the middle portion, and an upper portion located at a top end of the middle portion.

### Solution

According to an example embodiment, there is provided a microneedle in a structure of three or more layers, the microneedle including a middle portion configured to penetrate into the skin and formed of a compound containing a drug component; a lower portion configured to support the middle portion and including an inner pillar shell having a hollow central portion with a preset radius; and an upper portion provided at a top end of the middle portion and configured to facilitate penetration.

The lower portion may include a single inner pillar shell or a plurality of inner pillar shells each formed with a preset radius and at a height of the lower portion and having the hollow central portion.

The inner pillar shell may represent a core portion in a circular shape, an oval shape, a triangular shape, a quadrangular shape, or a polygonal shape.

The lower portion may have a donut shape or a porous shape based on a size of the inner pillar shell and a number of inner pillar shells.

The upper portion and the middle portion may have a pyramidal shape or a conical shape, and the lower portion may have a pyramidal shape or a cylindrical shape.

According to an example embodiment, there is provided a microneedle in a structure of three or more layers including a three-dimensional (3D) structure shell, the microneedle including a middle portion configured to penetrate into the skin and formed of a compound containing a drug component; a lower portion configured to support the middle portion and including a 3D structure shell in which a plurality of units including a plurality of straight members extending in different directions are coupled; and an upper portion provided at a top end of the middle portion and configured to facilitate penetration.

The lower portion may represent the 3D structure shell that is a truss structure form in which a unit in which the plurality of straight members extending in different directions are arranged in a triangular shape is coupled and the plurality of units connected in the triangular shape are stacked.

The lower portion may be configured to maintain a space between the plurality of straight members coupled as the unit and in the 3D structure shell and to adjust a melting rate after penetration into the skin by adjusting the space.

The upper portion and the middle portion may have a pyramidal shape or a conical shape.

A bottom diameter of the middle portion may be greater than a bottom diameter of the upper portion or a bottom diameter of the lower portion, and the bottom diameter of the upper portion may be greater than the bottom diameter of the lower portion.

According to an example embodiment, there is provided a microneedle containing a solid drug in a structure of three or more layers, the microneedle including a middle portion configured to penetrate into the skin and containing a solid drug in a cavity; a lower portion configured to support the middle portion; and an upper portion provided at a top end of the middle portion and configured to facilitate penetration.

The middle portion may include a cavity in a groove form with a predetermined size therein and includes, in the cavity, the solid drug in a structure in which a drug containing a drug is contained.

The middle portion may be configured to seal the solid drug by blocking a top of the cavity that includes the solid drug.

The middle portion may include a plurality of solid drugs including different drugs.

A cavity surface in contact with the solid drug may be coated with a waterproof material that does not react to the solid drug.

### Effect

According to example embodiments, by manufacturing a microneedle in a structure of three or more layers including a lower portion formed by including an inner pillar shell, it is possible to minimize a weight of the microneedle due to the hollow (inside-empty) inner pillar shell, to increase a melting rate of the lower portion due to an increase in a surface area, and to maintain strength. Further, according to example embodiments, in the microneedle in the structure of three or more layers, a melting rate that melts inside the skin may be adjusted based on a diameter size of the inner pillar shell and a number of pillars formed in a cross section.

According to example embodiments, by manufacturing a microneedle in a structure of three or more layers including a lower portion formed using a three-dimensional (3D) structure shell in which a plurality of straight members extending in different directions are coupled or a 3D structure shell in a pillar shape formed as a closed curved surface having a character-shaped cross section, it is possible to minimize a weight of the microneedle, to increase a melting rate of the lower portion due to an increase in a surface area, and to maintain strength. Further, according to example embodiments, in the microneedle in the structure of three or more layers, a melting rate that melts inside the skin may be adjusted based on a size, a height, a volume, and a shape of a 3D structure shell.

Also, according to example embodiments, by manufacturing a microneedle in a structure of three or more layers including a middle portion containing a solid drug, it is possible to enhance preservation of a drug contained in the middle portion and to facilitate penetration into the skin through an upper portion.

Also, according to example embodiments, by manufacturing a microneedle in a structure of three or more layers using a 3D printing technique, there are some advantages compared to an existing method in terms of technical aspect and boundary aspect, such as skin puncture, presence/absence of pain, the number density of needles, attachment time, precision, price, and scalability.

Also, in the case of manufacturing a microneedle according to the present invention, it is possible to secure high competitiveness in wrinkle-improving cosmetics market and medical market.

That is, the present invention is suitable for medical use since it is possible to manufacture a microneedle in a structure of three or more layers including an upper portion, a middle portion, and a lower portion in different forms.

### BRIEF DESCRIPTION OF DRAWINGS

FIG. 1 illustrates a perspective view of a microneedle according to an example embodiment.

FIGS. 2, 3A and 3B illustrate cross-sectional views of a microneedle according to an example embodiment.

FIGS. 4A and 4B illustrate cross-sectional views to describe a structural characteristic of a microneedle according to an example embodiment.

FIG. 5 illustrates an example of comparing a conventional method and a microneedle manufactured using a method according to the present invention.

FIG. 6 illustrates a perspective view of a microneedle patch manufactured according to an example embodiment.

FIG. 7 illustrates a flowchart of a microneedle manufacturing method according to an example embodiment.

FIG. 8 illustrates a process of manufacturing a microneedle using a microneedle manufacturing method according to an example embodiment.

FIG. 9 illustrates a perspective view of a microneedle including a three-dimensional (3D) structure shell according to an example embodiment.

FIG. 10 illustrates a cross-sectional view of a microneedle including a 3D structure shell according to an example embodiment.

FIG. 11 illustrates a cross-sectional view of a microneedle including a 3D structure shell according to another example embodiment.

FIGS. 12A and 12B illustrate cross-sectional views to describe a structural characteristic of a microneedle according to an example embodiment.

FIG. 13 illustrates an example of comparing a conventional method and a microneedle manufactured using a method according to the present invention.

FIG. 14 illustrates a perspective view of a microneedle patch manufactured according to an example embodiment.

FIG. 15 illustrates a flowchart of a method of manufacturing a microneedle including a 3D structure shell according to an example embodiment.

FIG. 16 illustrates a process of manufacturing a microneedle using a method of manufacturing a microneedle including a 3D structure shell according to an example embodiment.

FIG. 17 illustrates a flowchart of a method of manufacturing a microneedle including a 3D structure shell according to another example embodiment.

FIG. 18 illustrates a process of manufacturing a microneedle using a method of manufacturing a microneedle including a 3D structure shell according to another example embodiment.

FIG. 19 illustrates a perspective view of a microneedle containing a solid drug according to an example embodiment.

FIGS. 20A and 20B illustrate cross-sectional views of a microneedle containing a solid drug according to an example embodiment.

FIG. 21 illustrates a cross-sectional view of a microneedle containing a plurality of solid drugs according to an example embodiment.

FIG. 22 illustrates a cross-sectional view of a microneedle in a structure of three or more layers containing a solid drug according to an example embodiment.

FIG. 23 illustrates an example of comparing a conventional method and a microneedle manufactured using a method according to the present invention.

FIG. 24 illustrates a perspective view of a microneedle patch manufactured according to an example embodiment.

FIG. 25 illustrates a flowchart of a method of manufacturing a microneedle containing a solid drug according to an example embodiment.

FIG. 26 illustrates a process of manufacturing a microneedle containing a solid drug using a method of manufacturing the microneedle containing the solid drug according to an example embodiment.

### BEST MODE

Hereinafter, example embodiments are described in detail with reference to the accompanying drawings. However, the present invention is not limited to or restricted by the example embodiments. Also, like reference numerals in the respective drawings refer to like elements.

Also, terminologies used herein refer to terminologies used to appropriately represent example embodiments and may vary depending on the intent of a viewer and an operator or custom of a field to which the present invention pertains. Accordingly, the definition of the terms should be made based on the content throughout the specification.

The example embodiments may enhance preservation of a drug, may facilitate penetration into the skin, may increase a melting rate due to an increase in a surface area by a hollow inner pillar shell, and may maintain strength by manufacturing a microneedle in a structure of three or more layers including a middle portion formed of a compound containing a drug component, an upper portion provided at a top end of the middle portion and configured to facilitate penetration into the skin, and a lower portion configured to support the middle portion and including the inner pillar shell. Here, the microneedle according to the example embodiment is in a structure of three or more layers.

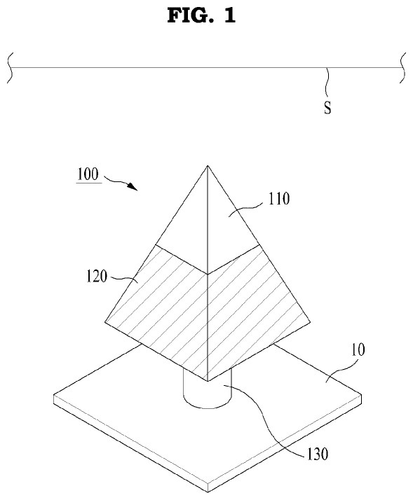

Hereinafter, example embodiments are described with reference to FIGS. 1 to 8.

FIG. 1 illustrates a perspective view of a microneedle according to an example embodiment.

Referring to FIG. 1, a microneedle 100 according to an example embodiment includes an upper portion 110, a middle portion 120, and a lower portion 130.

The upper portion 110 is provided at a top end of the middle portion 120 and facilitates penetration into the skin S. The upper portion 110 may have a sharp tip shape based on a penetration direction for penetrating into the skin S. For example, the upper portion 110 may be formed in a pyramidal shape or a conical shape such as a triangular shape, a quadrangular shape, a pentagonal shape, and a hexagonal shape, thereby facilitating penetration into the skin S. Here, to easily perforate the skin S, the upper portion 110 may be formed of a stronger material than those of the middle portion 120 and the lower portion 130.

The upper portion 110 according to an example embodiment allows the microneedle 100 to easily penetrate into the skin S and may protect the middle portion 120 formed of a compound containing a drug component.

According to an example embodiment, the upper portion 110 may be formed of a water-soluble substance that penetrates into the skin S and melts. For example, the water-soluble substance may be at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropyl methylcellulose (HPMC), ethylcellulose (EC), hydroxypropylcellulose (HPC), carboxymethylcellulose, cyclodextrin, and gentiobiose.

The middle portion 120 is capable of penetrating into the skin S through the upper portion 110 and is formed of a compound containing a drug component. The middle portion 120 is formed of the compound containing the drug component and is solidified. Therefore, when the middle portion 120 penetrates into the skin S through the upper portion 110, the solidified drug component may melt and be absorbed into the skin S.

Although the middle portion 120 of the microneedle 100 according to an example embodiment is formed of a compound containing a drug component, that is, solidified, the middle portion 120 may include a cavity capable of containing a drug in a liquid state depending on example embodiments.

The middle portion 120 is in a truncated pyramidal shape or a truncated conical shape, such as a triangle, a square, a pentagon, and a hexagon from which the upper portion 110 is removed, and may include a cavity area in which a drug is containable. Here, the drug may be solidified. The cavity area may be provided in an upper area above the center of the middle portion 120. However, depending on example embodiments, a position, a size, and a shape of the cavity area may be variously applied based on an administration point in time, an administration time, and an administration amount of the drug. Further, a size and a position of the cavity may be adjusted based on an amount, an evaporation rate, and a temperature of the drug, a shape of the middle portion 120 for manufacture of the microneedle 100, viscosity of the drug, concentration of the drug, a solvent used, and a thickness that covers a top of the cavity.

Similar to the upper portion 110 that penetrates into the skin S, the middle portion 120 may be formed of a water-soluble substance. Here, the middle portion 120 is formed of a compound containing a drug component and may be formed of a substance different from those of the upper portion 110 and the lower portion 130.

Here, the drug component of the middle portion 120 may be formed using a biocompatible material and an additive. For example, the biocompatible material may include at least one of carboxymethylcellulose (CMC), hyaluronic acid (HA), alginic acid, pectin, carrageenan, chondroitin sulfate, dextran sulfate, chitosan, polylysine, carboxymethyl chitin, fibrin, agarose, pullulan, polyanhydride, polyorthoester, polyetherester, polyesteramide, poly butyric acid, poly valeric acid, polyacrylate, ethylene-vinyl acetate polymer, acrylic-substituted cellulose acetate, polyvinyl chloride, polyvinyl fluoride, polyvinyl imidazole, chlorosulphonate polyolefins, polyethylene oxide, polyvinylpyrrolidone (PVP), hydroxypropyl methylcellulose (HPMC), ethylcellulose (EC), hydroxypropylcellulose (HPC), carboxymethyl cellulose, cyclodextrin, maltose, lactose, trehalose, cellobiose, isomaltose, turanose, and lactulose, or may include at least one of a copolymer of monomers that form such the polymer and cellulose.

Also, the additive may include at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropyl methylcellulose (HPMC), ethylcellulose (EC), hydroxypropylcellulose (HPC), carboxymethyl cellulose, cyclodextrin, gentiobiose, alkyltrimethylammonium bromide (cetrimide), hexadecyltrimethylammonium bromide (CTAB), gentian violet, benzethonium chloride, docusate sodium salt, a SPAN-type surfactant, polysorbate (Tween), sodium dodecyl sulfate (SDS), benzalkonium chloride, and glyceryl oleate.

Also, the drug component of the middle portion 120 may be formed by mixing a biocompatible material and an active ingredient. The active ingredient includes protein/peptide medicine. However, it is provided as an example only and the active ingredient includes at least one of hormone, hormone analog, enzyme, enzyme inhibitor, signal transduction protein or a portion thereof, antibody or a portion thereof, single-chain antibody, binding protein or a binding domain thereof, antigen, adhesion protection, structural protein, regulatory protein, toxin protein, cytokine, a transcription factor, a blood coagulation factor, and vaccine. In detail, the protein/peptide medicine may include one of insulin, 9nsulin-like growth factor 1 (IGF-1), growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), a tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormone releasing hormone (LHRH), nafarelin, parathormone, pramlintide, T-20 (enfuvirtide), thymalfasin, and ziconotide.

Also, a solvent of the drug component of the middle portion 120 may dissolve the biocompatible material. The solvent may include at least one of inorganic and organic solvents that include DI water, methanol, ethanol, chloroform, dibutyl phthalate, dimethyl phthalate, ethyl lactate, glycerin, isopropyl alcohol, lactic acid, propylene glycol, and the like.

The microneedle 100 according to an example embodiment may administer a quantitative drug by forming a cavity of a specific area in the middle portion 120 and by containing a drug in a liquid state in the cavity. Accordingly, the present invention may enhance preservation of the drug, may facilitate penetration into the skin, and may administer the drug in the liquid state.

The lower portion 130 supports the middle portion 120 and includes an inner pillar shell having a hollow central portion with a preset radius. The lower portion 130 may include a single inner pillar shell or a plurality of inner pillar shells in an outer shell of a pyramidal shape or a cylindrical shape, such as a triangle, a square, a pentagon, and a hexagon.

The lower portion 130 has a predetermined diameter and height, and may represent a depth of the microneedle 100 that penetrates into the skin S. For example, a penetration depth of the upper portion 110 and the middle portion 120 containing a drug into the skin S may be estimated based on the diameter and the height of the lower portion 130. The height of the lower portion 130 may be adjusted based on a type of the drug, a state of the drug, an administration point in time of the drug, an administration time of the drug, and an administration amount of the drug. Also, a diameter of the outer shell of the lower portion 130 may be adjusted based on a weight and a size of the upper portion 110 and the middle portion 120, a level of sustaining the drug, and an amount of time used for the lower portion 130 to melt in the skin S.

The lower portion 130 is formed of a melting material that connects a base portion 10 and the microneedle 100, and separates the microneedle 100 from the base portion 10. For example, the lower portion 130 is formed of a water-soluble substance and may penetrate into the skin S and quickly melt, thereby rapidly separating the microneedle 100 formed on the base portion 10.

Here, similar to the upper portion 110 and the middle portion 120 that penetrate into the skin S, the lower portion 130 may be formed of a water-soluble substance. Here, the lower portion 130 may be formed of a substance that further quickly melts than those of the upper portion 110 and the middle portion 120 among water-soluble substances. The upper portion 110 is to further facilitate skin puncture, the middle portion 120 is to further efficiently administer a drug, and the lower portion 130 is to quickly separate the microneedle 100 formed on the base portion 10 and to estimate a penetration depth of the microneedle 100 into the skin S. Therefore, the microneedle 100 according to an example embodiment includes the upper portion 110, the middle portion 120, and the lower portion 130 in a structure of three or more layers formed of different substances.

The lower portion 130 according to an example embodiment may include a single inner pillar shell or a plurality of inner pillar shells each formed with a preset radius and at a height of the lower portion 130 and having a hollow central portion in the outer shell with a preset diameter and height. For example, the inner pillar shell is formed with a shaped cross section, such as a circular shape, an oval shape, a triangular shape, a quadrangular shape, or a polygonal shape, and at the same height as the height of the lower portion 130 and, depending on example embodiments to which the present invention applies, a single inner pillar shell or a plurality of inner pillar shells may be formed. Therefore, the lower portion 130 may have a donut shape or a porous shape based on a size and a number of inner pillar shells.

Further, a diameter size and a number of inner pillar shells formed on the cross section of the lower portion 130 may be adjusted based on a depth of the lower portion 130 that penetrates into the skin S, a melting rate thereof, and a type of a drug substance. The microneedle 100 according to an example embodiment may minimize a weight of the microneedle 100 by including the lower portion 130 formed including a single inner pillar shell or the plurality of inner pillar shells, may increase a melting rate of the lower portion 130 due to an increase in a surface area caused by the inner pillar shell, and may maintain strength of the lower portion 130 by manufacturing the lower portion 130 that includes a hollow central portion, but is structurally stabilized.

Also, the lower portion 130 according to an example embodiment may represent a depth of penetration into the skin as a role of supporting the upper portion 110 and the middle portion 120 in the microneedle 100. Referring to FIG. 1, the lower portion 130 is in a pyramidal shape or a cylindrical shape and has a small size and volume compared to those of the upper portion 110 and the middle portion 120. Accordingly, the lower portion 130 may minimize an area, volume, and weight of the microneedle 100 and may support a quantitative drug to be administered due to a shape of an appropriate size, height, and diameter according to a depth of the microneedle 100 that penetrates into the skin S.

Depending on example embodiments, the microneedle 100 according to an example embodiment may include the upper portion 110 and the middle portion 120 formed by including an inner shell formed in a pyramidal shape or a conical shape, such as a triangle, a square, a pentagon, and a hexagon, as well as the lower portion 130.

Depending on other example embodiments, the lower portion 130 of the microneedle 100 according to an example embodiment may be formed using a 3D structure shell that is in a structure in which a plurality of straight members extending in different directions are coupled or in a structure of a pillar shape formed as a closed curved surface having a character-shaped cross section.

Referring again to FIG. 1, the microneedle 100 may be formed on the base portion 10. A drug is not provided to the base portion 10, and the base portion 10 is separable after the microneedle 100 including the upper portion 110, the middle portion 120, and the lower portion 130 penetrates into the skin S. For example, the base portion 10 may be provided in a patch form and may closely attach to the skin S.

Dissimilar to the microneedle 100 that penetrates into the skin S, the base portion 10 may be formed of a non-water-soluble substance that does not melt. Therefore, the base portion 10 may guide feed of a quantitative drug contained in the middle portion 120 by not interfering with the penetrating power of the microneedle 100.

For example, the base portion 10 may be formed of at least one from a group including polyethylene (PE), polypropylene (PP), polytetrafluoroethylene (PTFE), polymethyl methacrylate (PMMA), ethylene vinyl acetate (EVA), polycaprolactone (PCL), polyurethane (PU), polyethylene terephthalate (PET), polyethylene glycol (PEG), polyvinyl alcohol (PVA), polylactide (PLA), polylactide-glycolide copolymer (PLGA), and polyglycolide (PGA).

Referring to FIG. 1, the microneedle 100 according to an example embodiment may form, in a tree-shaped structure of three or more layers, the middle portion 120 formed of a compound containing a drug component, the upper portion 110 provided at a top end of the middle portion 120 and configured to facilitate penetration into the skin S, and the lower portion 130 configured to support the middle portion 120 and to facilitate separation from the base portion 10, thereby enhancing preservation of the drug, facilitating penetration into the skin, enabling quantitative administration of the drug, and increasing a melting rate due to an increase in a surface area.

Also, the microneedle 100 according to an example embodiment is in a tree-shaped structure of three or more layers and thus, it is possible to increase a penetration rate (60% or more) of the structure and an absorption rate of useful components into the skin by minimizing penetration resistance caused by skin elasticity when attaching to the skin. Also, the tree-shaped microneedle 100 applies the structure of three or more layers to maximize mechanical strength of the structure and facilitating penetration into the skin.

Also, the upper portion 110 and the middle portion 120 each in a conical shape or a pyramidal shape and the lower portion 130 in a pyramidal shape or a cylindrical shape that form the microneedle 100 according to an example embodiment are manufactured using a 3D printing technique. Since the present invention employs a 3D printing scheme, it is possible to acquire a very short attachment time, high precision, and low price compared to the conventional method. Also, it is possible to increase the number density of microneedles 100 in a micro patch and to improve an aspect ratio.

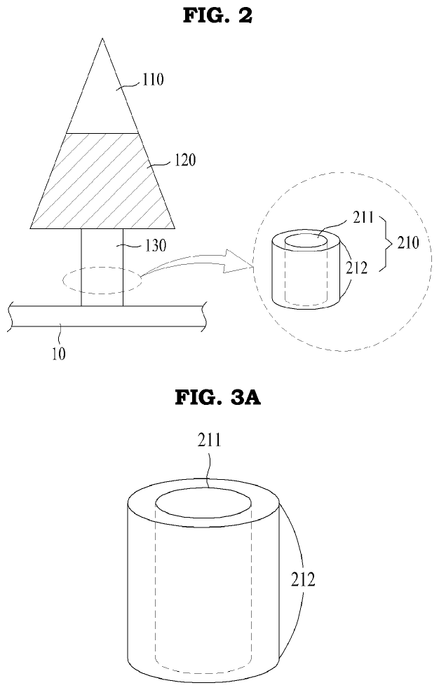

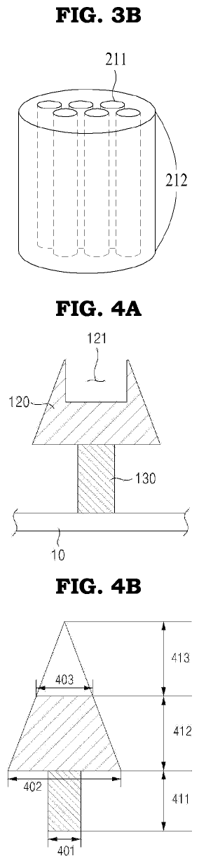

FIGS. 2, 3A and 3B illustrate cross-sectional views of a microneedle according to an example embodiment.

Referring to FIGS. 2, 3A and 3B, the microneedle 100 includes the lower portion 130 including an inner pillar shell 210, the middle portion 120, and the upper portion 110.

A cross section of the lower portion 130 of the microneedle 100 according to an example embodiment may be in a pyramidal shape or a cylindrical shape, such as a triangle, a square, a pentagon, and a hexagon. A core portion 211 that forms the inner pillar shell 210 may have a shaped cross section in various polygonal shapes, such as a circular shape, an oval shape, a triangular shape, a quadrangular shape, and a pentagonal shape.

Referring to a cross-sectional view of the lower portion 130 of FIG. 2, the inner pillar shell 210 is in a form in which a central portion of the lower portion 130, that is, the core portion 211 is hollow (empty) and formed with a preset radius and provided inside the lower portion 130. Here, a shape, a size, a number, and arrangement for the inner pillar shell 210 with the same height 212 as that of the lower portion 130 based on the core portion 211 may be arbitrarily adjusted and used. For example, a diameter, an arrangement, and a number for the inner pillar shell 210 formed in the lower portion 130 may be designated into consideration of a type of a drug contained in the microneedle 100, a state of the drug, an administration point in time of the drug, an administration time of the drug, an administration amount of the drug, and a melting time of the drug.

Referring to FIGS. 3A and 3B, the lower portion 130 of the microneedle 100 according to an example embodiment may have a donut shape (a) or a porous shape (b) based on a size and a number of inner pillar shells 210.

The donut shape FIG. 3A has the core portion 211 with a size greater than that of the porous shape FIG. 3B and may be provided in a form in which a single core portion 211 is formed in a central portion of the lower portion 130. Also, the porous shape FIG. 3B has the core portion 211 with a size less than that of the donut shape FIG. 3A and may be provided in a form in which a plurality of core portions 211 are formed in the lower portion 130.

According to an example embodiment, although FIGS. 3A and 3B illustrates that the inner pillar shell 210 has a hollow central portion (core portion 211) with a preset radius and has the same height 212 as that of the lower portion 130, the height of the inner pillar shell 210 may differ from that of the lower portion 130. Further, the lower portion 130 may be in a form in which the plurality of inner pillar shells 210 each having a different radius and height are stacked through various arrangement.

FIGS. 4A and 4B illustrate cross-sectional views to describe a structural characteristic of a microneedle according to an example embodiment.

In detail, FIG. 4A illustrates a cross-sectional view of a microneedle including a cavity according to an example embodiment and FIG. 4B illustrates a cross-sectional view of a microneedle in a structure of three or more layers according to an example embodiment.

The microneedle 100 according to an example embodiment may basically include the middle portion 120 formed of a compound containing a drug component, that is, a solidified material, and, depending on example embodiments, may also include the middle portion 120 in which a cavity 121 is formed to contain a drug in a liquid state.

Referring to FIG. 4A, the microneedle 100 according to an example embodiment may include the middle portion 120 that includes the cavity 121. The cavity 121 may be formed in a groove form within the middle portion 120 and may be formed in a shape and a size for containing a drug.

Here, the drug to be contained in the cavity 121 may be a liquid drug to be injected and may also be a solid drug (not shown) in a form of a capsule (micro-sphere) depending on example embodiments. For example, the solid drug may be a polyhedron, such as a circular shape, an oval shape, a capsule type, a hexahedron, and a square prism, and may be formed using different sizes and shapes based on a type of a drug that penetrates into and acts on the skin S, strength of the drug, intensity of the drug, a state of the drug, an administration point in time of the drug, an administration time of the drug, an administration amount of the drug, and a subject of the drug.

Here, the cavity surface to contact the drug may be coated with a waterproof material. When the microneedle 100 includes the cavity 121, the microneedle 100 may contain a drug in a liquid state. Therefore, the drug may be absorbed in the middle portion 120 and thus, to prevent this, the cavity surface is coated with the waterproof material.

For example, the cavity surface may be coated with a waterproof agent that includes a mineral-based material or a lipid-based material. Here, the waterproof agent may include at least one of beeswax, oleicacid, soy fatty acid, castor oil, phosphatidylcholine, d-α-tocopherol/vitamin E, corn oil mono-ditridiglycerides, cottonseed oil, olive oil, peanut oil, peppermint oil, safflower oil, sesame oil, soybean oil, hydrogenated vegetable oils, hydrogenated soybean oil, caprylic/capric triglycerides derived from coconut oil or palm see oil, and phosphatidylcholine, or may be formed using a mixture thereof.

Depending on example embodiments, the cavity surface may be coated with a different waterproof agent based on a type and a state of a drug to be injected into the cavity 121 and the cavity 121 may be formed in the middle portion 120 to have a different size, height, and shape based on a type of the drug, a state of the drug, an administration point in time of the drug, an administration time of the drug, and an administration amount of the drug.

Referring to FIG. 4B, the microneedle 100 according to an example embodiment is a microstructure configured in a structure of three or more layers and includes the upper portion 110 and the middle portion 120 each in a pyramidal shape or a conical shape and the lower portion 130 in a pyramidal shape or a cylindrical shape.

Referring to FIG. 4B, a bottom diameter 402 of the middle portion 120 is greater than a bottom diameter 403 of the upper portion 110 or a bottom diameter 401 of the lower portion 130, and the bottom diameter 403 of the upper portion 110 is greater than the bottom diameter 401 of the lower portion 130. Sizes may be determined in order of the bottom diameter 402 of the middle portion 120, the bottom diameter 403 of the upper portion 110, and the bottom diameter 401 of the lower portion 130. Here, the bottom diameter 401 of the lower portion 130 refers to a diameter of an outer shell that includes an inner pillar shell.

Also, a height 412 of the middle portion 120 may be greater than a height 413 of the upper portion 110 and an addition of the height 412 of the middle portion 120 and the height 413 of the upper portion 110 may be greater than or less than a height 411 of the lower portion 130. That is, in the microneedle 100 according to an example embodiment, the height 412 of the middle portion 120 may be highest and the height 413 of the upper portion 110 may be equal to the height 411 of the lower portion 130, which may differ depending on example embodiments to which the microneedle 100 according to an example embodiment applies. Here, the height 411 of the lower portion 130, the height 412 of the middle portion 120, and the height 413 of the upper portion 110 in the microneedle 100 according to an example embodiment are not limited to FIG. 4B and may be variously defined depending on example embodiments.

Since the cavity 121 for containing a drug is formed in the middle portion 120 of the microneedle 100 according to an example embodiment, the middle portion 120 may be formed with the largest volume, the largest bottom diameter 402, and the highest height 412. The upper portion 110 may be in a pyramidal shape or a conical shape to penetrate into the skin S and the bottom diameter 403 of the upper portion 110 is equal to a top (or a tip) of the middle portion 120 and may be determined based on a width of a cross-sectional area of a tip of a truncated pyramid or a truncated cone that forms the middle portion 120. Also, the height 413 of the upper portion 110 may be determined based on a shape of the truncated pyramid or the truncated cone of the middle portion 120.

The lower portion 130 of the microneedle 100 according to an example embodiment functions to support the upper portion 110 and the middle portion 120 in the microneedle 100 and may represent a depth of penetration into the skin. Therefore, the volume and the bottom diameter 401 of the lower portion 130 may be less than those of the upper portion 110 and the middle portion 120. Here, the height 411 of the lower portion 130 may be determined based on the depth of penetration into the skin.

The lower portion 130 is in a pyramidal shape or a cylindrical shape and includes the bottom diameter 401 less than the bottom diameter 403 of the upper portion 110 and the bottom diameter 402 of the middle portion 120, and has the volume less than those of the upper portion 110 and the middle portion 120. The lower portion 130 represents a depth of the microneedle 100 that penetrates into the skin S and is configured to support the upper portion 110 and the middle portion 120 and thus, minimizes an area, volume, and weight of the microneedle 100 according to an example embodiment. Accordingly, the lower portion 130 may support a liquid drug to be quantitatively administered due to an appropriate size, height, and diameter according to the depth of the microneedle 100 that penetrates into the skin S.

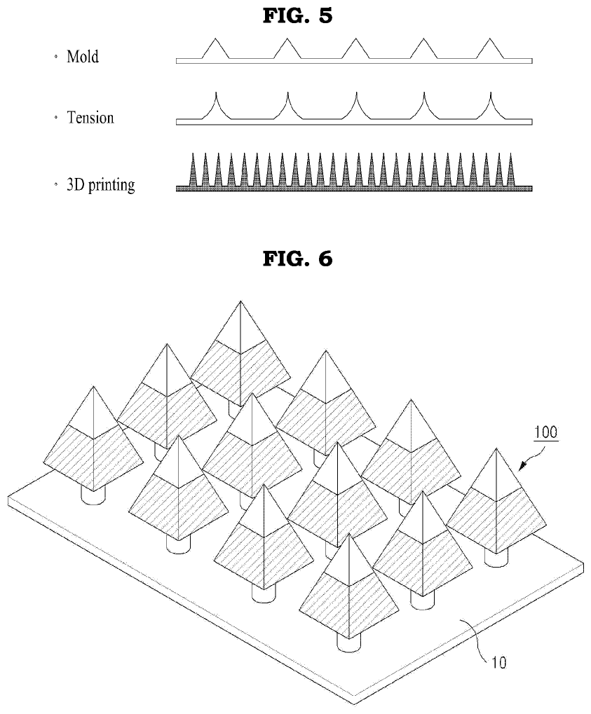

FIG. 5 illustrates an example of comparing a conventional method and a microneedle manufactured using a method according to the present invention, and FIG. 6 illustrates a perspective view of a microneedle patch manufactured according to an example embodiment.

Referring to FIG. 5, while a mold scheme and a tension scheme exhibit the low number density of microneedles, a microneedle according to an example embodiment manufactured using a lamination scheme, for example, a 3D printing scheme exhibits the very high number density compared to the conventional method in which the number density is low due to limitations found in the mold scheme and the tension scheme. Also, the microneedle manufactured by the method according to the present invention has a higher aspect ratio compared to the mold scheme and the tension scheme. Also, the method according to the present invention may adjust the aspect ratio of the microneedle and the aspect ratio may be determined based on a field in which the microneedle of the present invention is used, for example, a field according to treatment use and medical use.

The method (3D printing) according to the present invention is advantageous in terms of skin puncture and painless compared to the mold scheme and has the high number density of microneedles compared to the mold scheme and the tension scheme. Also, compared to the conventional method, the method according to the present invention may acquire a very short attachment time and high precision. Since a lamination scheme, for example, a 3D printing scheme is used, manufacturing price is low and scalability is high accordingly. As described above, the method according to the present invention is very advantageous in technical and economical aspects compared to the conventional method, for example, the mold scheme and the tension scheme.

That is, since the microneedle implemented by a lamination technique with the method according to the present invention has a high aspect ratio, skin puncture is excellent, pain is very low, and the number density is high, which leads to significantly decreasing an attachment time. In addition, the present invention may implement the microneedle with high precision of about 5 micrometers and may place a desired drug at a desired position, thereby acquiring high scalability.

Referring to FIG. 6, the microneedle 100 manufactured as described above may be provided as a microneedle patch in which a plurality of microneedles 100 are formed on the base portion 10 and may be easily applied to a medical field. That is, the present invention may secure high competitiveness in a medical market field by manufacturing the microneedle 100 in a structure of three or more layers by applying a lamination scheme using 3D printing.

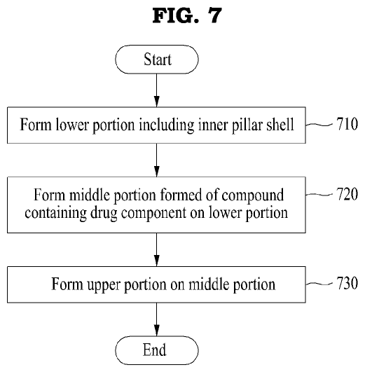

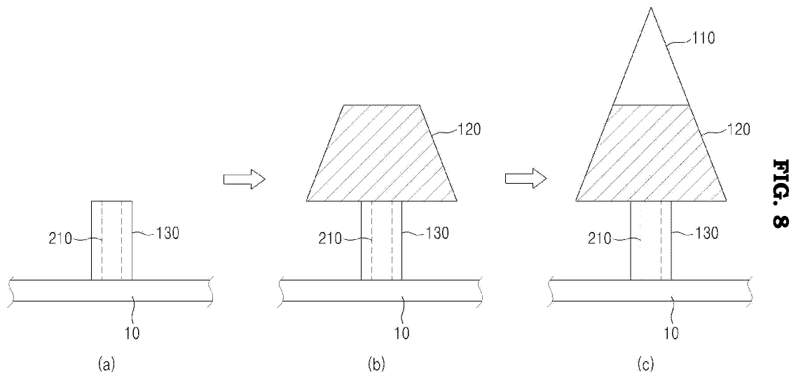

FIG. 7 illustrates a flowchart of a microneedle manufacturing method according to an example embodiment, and FIG. 8 illustrates a process of manufacturing a microneedle using a microneedle manufacturing method according to an example embodiment.

The microneedle 100 of FIG. 8 manufactured through the manufacturing method of FIG. 7 may be manufactured using a 3D printing scheme.

Referring to FIG. 7 and (a) of FIG. 8, in operation 710, the lower portion 130 including the inner pillar shell 210 having a hollow central portion with a preset radius is formed. The microneedle manufacturing method according to an example embodiment may form, on the base portion 10, the lower portion 130 that includes a single inner pillar shell 210 or the plurality of inner pillar shells 210 in an outer shell in a pyramidal shape or a cylindrical shape, such as a triangle, a square, a pentagon, and a hexagon.

The lower portion 130 may have a predetermined diameter and height, and may represent a depth of the microneedle 100 that penetrates into the skin S. For example, a penetration depth of the upper portion 110 and the middle portion 120 containing a drug into the skin S may be estimated based on the diameter and the height of the lower portion 130, and the height of the lower portion 130 according to a depth of a drug to be penetrated may be adjusted based on a type of the drug, a state of the drug, an administration point in time of the drug, an administration time of the drug, and an administration amount of the drug.

A diameter of the outer shell of the lower portion 130 may be adjusted based on a weight and a size of the upper portion 110 and the middle portion 120, a level of sustaining the drug, and an amount of time used for the lower portion 130 to melt in the skin S and a radius and a number of inner pillar shells 210.

For example, the lower portion 130 according to an example embodiment may include a single inner pillar shell 210 or the plurality of inner pillar shells 210 each formed with a preset radius and at a height of the lower portion 130 and having a hollow central portion in the outer shell with a predetermined diameter and height. The inner pillar shell 210 may be formed with a shaped cross section such as a circular shape, an oval shape, a triangular shape, a quadrangular shape, or a polygonal shape and at the same height as the height of the lower portion 130. The lower portion 130 may have a donut shape or a porous shape based on a size and a number of inner pillar shells 210.

Further, a diameter size and a number of inner pillar shells 210 formed on the cross section of the lower portion 130 may be adjusted based on a depth of the lower portion 130 that penetrates into the skin S, a melting rate thereof, and a type of a drug substance. The microneedle 100 according to an example embodiment may minimize a weight of the microneedle 100 by including the lower portion 130 formed including a single inner pillar shell 210 or the plurality of inner pillar shells 210 each having a hollow (empty) inside, may increase a melting rate of the lower portion 130 due to an increase in a surface area caused by the inner pillar shell 210, and may maintain strength of the lower portion 130 by manufacturing the lower portion 130 that includes a hollow central portion, but is structurally stabilized.

The lower portion 130 is formed of a melting material that connects the base portion 10 and the microneedle 100, and separates the microneedle 100 from the base portion 10. For example, the lower portion 130 may be formed of a water-soluble substance and may penetrate into the skin S and quickly melt, thereby rapidly separating the microneedle 100 formed on the base portion 10.

Here, similar to the upper portion 110 and the middle portion 120 that penetrate into the skin S, the lower portion 130 may be formed of a water-soluble substance. Here, the lower portion 130 may be formed of a substance that further quickly melts than those of the upper portion 110 and the middle portion 120 among water-soluble substances. Here, the upper portion 110 is to further facilitate skin puncture, the middle portion 120 is to further efficiently administer the drug, and the lower portion 130 is to quickly separate the microneedle 100 formed on the base portion 10 and to estimate a penetration depth of the microneedle 100 into the skin S. Therefore, the microneedle 100 according to an example embodiment includes the upper portion 110, the middle portion 120, and the lower portion 130 in a structure of three or more layers formed of different substances.

In operation 720, the middle portion 120 that penetrates into the skin and formed of a compound containing a drug component is formed on the lower portion 130. Referring to (b) of FIG. 8, the microneedle manufacturing method according to an example embodiment may form, on the lower portion 130, the solidified middle portion 120 formed of a compound containing a drug component. Here, although (b) of FIG. 8 illustrates the middle portion 120 formed of the compound containing the drug component, the middle portion 120 of the microneedle 100 according to an example embodiment may include a cavity capable of containing a drug in a liquid state.

According to an example embodiment, the middle portion 120 may be formed of a water-soluble substance, which is similar to the upper portion 110 that penetrates into the skin S. Here, since the middle portion 120 is formed of the compound containing the drug component, the middle portion 120 may be formed of a substance different from those of the upper portion 110 and the lower portion 130.

In operation 730, the upper portion 110 is formed on the middle portion 120. Referring to (c) of FIG. 8, the microneedle manufacturing method according to an example embodiment may form the upper portion 110 provided at a top end of the middle portion 120 and configured to facilitate penetration into the skin S. The upper portion 110 may have a sharp tip shape based on a penetration direction for penetrating into the skin S. For example, the upper portion 110 may be formed in a pyramidal shape or a conical shape such as a triangular shape, a quadrangular shape, a pentagonal shape, and a hexagonal shape, thereby facilitating penetration into the skin S.

Each of the upper portion 110, the middle portion 120, and the lower portion 130 of the microneedle 100 according to an example embodiment may be formed of a different material. Although the upper portion 110, the middle portion 120, and the lower portion 130 may be formed of the same water-soluble substance, the upper portion 110 configured to facilitate penetration, the middle portion 120 formed of a compound containing a drug component, and the lower portion 130 configured to support the middle portion 120 and to facilitate separation from the base portion 10 may be formed of different types of substances among water-soluble substances.

Example embodiments are to enhance preservation of a drug, to facilitate penetration into the skin, to decrease a weight, to increase a melting rate due to an increase in a surface area caused by a 3D structure shell, and to maintain strength by manufacturing a microneedle in a structure of three or more layers including a middle portion formed of a compound containing a drug component, an upper portion provided at a top end of the middle portion and configured to facilitate penetration into the skin, and a lower portion configured to support the middle portion. Here, the microneedle according to an example embodiment is in the structure of three or more layers.

Also, the 3D structure shell according to an example embodiment is a structure in which a plurality of straight members extending in different directions are coupled or a structure of a pillar shape formed as a closed curved shape having a character-shaped cross section.

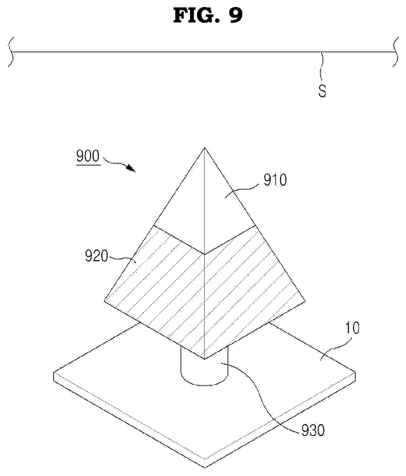

Hereinafter, example embodiments are described in detail with reference to FIGS. 9 to 18.

FIG. 9 illustrates a perspective view of a microneedle including a 3D structure shell according to an example embodiment.

Referring to FIG. 9, a microneedle 900 including a 3D structure shell according to an example embodiment includes an upper portion 910, a middle portion 920, and a lower portion 930.

The upper portion 910 is provided at a top end of the middle portion 920 and facilitates penetration into the skin S. The upper portion 910 may have a sharp tip shape based on a penetration direction into the skin S. For example, the upper portion 110 may be formed in a pyramidal shape or a conical shape such as a triangular shape, a quadrangular shape, a pentagonal shape, and a hexagonal shape, thereby facilitating penetration into the skin S. Here, to facilitate puncture of the skin S, the upper portion 910 may be formed of a material with stronger strength than those of the middle portion 920 and the lower portion 930.

The upper portion 910 according to an example embodiment allows the microneedle 900 to easily penetrate into the skin S and may protect the middle portion 920 formed of a compound containing a drug component.

Depending on example embodiments, the upper portion 910 may be formed of a water-soluble substance that penetrates into the skin S and melts. For example, the water-soluble substance may include at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropyl methylcellulose (HPMC), ethylcellulose (EC), hydroxypropylcellulose (HPC), carboxymethylcellulose, cyclodextrin, and gentiobiose.

The middle portion 920 is capable of penetrating into the skin S through the upper portion 910 and is formed of a compound containing a drug component. The middle portion 920 is formed of the compound containing the drug component and is solidified. Therefore, when the middle portion 920 penetrates into the skin S through the upper portion 910, the solidified drug component may melt and be absorbed into the skin S.

Although the middle portion 920 of the microneedle 900 including the 3D structure shell according to an example embodiment is formed of a compound containing a drug component, that is, solidified, the middle portion 920 may include a cavity capable of containing a drug in a liquid state depending on example embodiments.

The middle portion 920 is in a truncated pyramidal shape or a truncated conical shape, such as a triangle, a square, a pentagon, and a hexagon from which the upper portion 910 is removed, and may include a cavity area in which a drug is contained. Here, the drug may be solidified. The cavity area may be provided in an upper area above the center of the middle portion 920. However, depending on example embodiments, a position, a size, and a shape of the cavity area may be variously applied based on an administration point in time, an administration time, and an administration amount of the drug. Further, a size and a position of the cavity may be adjusted based on an amount, an evaporation rate, and a temperature of the drug, a shape of the middle portion 920 for manufacture of the microneedle 900, viscosity of the drug, concentration of the drug, a solvent used, and a thickness that covers a top of the cavity.

Similar to the upper portion 910 that penetrates into the skin S, the middle portion 920 may be formed of a water-soluble substance. Here, the middle portion 920 is formed of a compound containing a drug component and may be formed of a substance different from that of the upper portion 910 and the lower portion 930.

Here, the drug component of the middle portion 920 may be formed of a biocompatible material and an additive. For example, the biocompatible material may include at least one of carboxymethylcellulose (CMC), hyaluronic acid (HA), alginic acid, pectin, carrageenan, chondroitin sulfate, dextran sulfate, chitosan, polylysine, carboxymethyl chitin, fibrin, agarose, pullulan, polyanhydride, polyorthoester, polyetherester, polyesteramide, poly butyric acid, poly valeric acid, polyacrylate, ethylene-vinyl acetate polymer, acrylic-substituted cellulose acetate, polyvinyl chloride, polyvinyl fluoride, polyvinyl imidazole, chlorosulphonate polyolefins, polyethylene oxide, polyvinylpyrrolidone (PVP), hydroxypropyl methylcellulose (HPMC), ethylcellulose (EC), hydroxypropylcellulose (HPC), carboxymethyl cellulose, cyclodextrin, maltose, lactose, trehalose, cellobiose, isomaltose, turanose, and lactulose, or may include at least one of a copolymer of monomers that form such the polymer and cellulose.

Also, the additive may include at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropyl methylcellulose (HPMC), ethylcellulose (EC), hydroxypropylcellulose (HPC), carboxymethyl cellulose, cyclodextrin, gentiobiose, alkyltrimethylammonium bromide (cetrimide), hexadecyltrimethylammonium bromide (CTAB), gentian violet, benzethonium chloride, docusate sodium salt, a SPAN-type surfactant, polysorbate (Tween), sodium dodecyl sulfate (SDS), benzalkonium chloride, and glyceryl oleate.

Also, the drug component of the middle portion 920 may be formed by mixing a biocompatible material and an active ingredient. The active ingredient includes protein/peptide medicine. However, it is provided as an example only and the active ingredient includes at least one of hormone, hormone analog, enzyme, enzyme inhibitor, signal transduction protein or a portion thereof, antibody or a portion thereof, single-chain antibody, binding protein or a binding domain thereof, antigen, adhesion protection, structural protein, regulatory protein, toxin protein, cytokine, a transcription factor, a blood coagulation factor, and vaccine. In detail, the protein/peptide medicine may include one of insulin, insulin-like growth factor 1 (IGF-1), growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, Epidermal growth factors (EGFs), calcitonin, adrenocorticotropic hormone (ACTH), a tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormonereleasing hormone (LHRH), nafarelin, parathormone, pramlintide, T-20 (enfuvirtide), thymalfasin, and ziconotide.

Also, a solvent of the drug component of the middle portion 920 may dissolve the biocompatible material. The solvent may include at least one of inorganic and organic solvents that include DI water, methanol, ethanol, chloroform, dibutyl phthalate, dimethyl phthalate, ethyl lactate, glycerin, isopropyl alcohol, lactic acid, propylene glycol, and the like.

The microneedle 900 including the 3D structure shell according to an example embodiment may administer a quantitative drug by forming a cavity of a specific area in the middle portion 920 and by containing a drug in a liquid state in the cavity to penetrate into the skin S. Accordingly, the present invention may enhance preservation of the drug, may facilitate penetration into the skin, and may administer the drug in the liquid state.

The lower portion 930 supports the middle portion 920 and includes a 3D structure shell in which a plurality of units including a plurality of straight members extending in different directions are coupled or a 3D structure shell of a pillar shape formed as a closed curved surface having a character-shaped cross section.

The lower portion 930 may represent a depth of the microneedle 900 including the 3D structure shell that penetrates into the skin S. For example, a penetration depth of the upper portion 910 and the middle portion 920 containing the drug into the skin S may be estimated based on a height of the lower portion 930, and the height of the lower portion 930 according to a depth of the drug to be penetrated may be adjusted based on a type of the drug, a state of the drug, an administration point in time of the drug, an administration time of the drug, and an administration amount of the drug. Also, the lower portion 930 may need to support the weight and the size of the upper portion 910 and the middle portion 920 and the drug and to be structurally stable. More importantly, the lower portion 930 may need to adjust a melting rate. Further, the lower portion 930 may adjust a form of the 3D structure shell based on a melting degree and time after penetration into the skin S.

For example, the lower portion 930 may be formed in the 3D structure shell in which the plurality of units including the plurality of straight members extending in different directions are coupled or the 3D structure shell of the pillar shape formed as the closed curved surface having the character-shaped cross section. Here, a melting rate of the microneedle 900 may be improved due to an increase in a surface area by the 3D structure shell.

According to an example embodiment, the lower portion 930 may be in a 3D structure shell that is a truss structure form in which a unit in which a plurality of straight members extending in different directions are arranged in a triangular shape is coupled and a plurality of units connected in the triangular shape are stacked. Here, the lower portion 930 may maintain a space between the plurality of straight members coupled as a unit and in the 3D structure shell and may adjust a melting rate after penetration into the skin by adjusting the space.

According to another example embodiment, the lower portion 930 may be in a 3D structure shell that represents a pillar shape having a character-shaped cross section of an alphabetical character “C” or “H” or an irregular cross section and formed at a height of the lower portion 130. Here, the lower portion 930 is in a 3D structure of a closed curved surface form in which a space is maintained.

That is, the 3D structure shell may be applied to the lower portion 930 of the microneedle 900 including the 3D structure shell according to an example embodiment based on a depth of the lower portion 930 that penetrates into the skin S, a melting rate, and a type of a drug substance. The microneedle 900 including the 3D structure shell according to an example embodiment may include the lower portion 930 formed as the 3D structure shell and thereby minimize a weight of the microneedle 900, increase a melting rate of the lower portion 930 due to an increase in a surface area, and maintain strength using the structurally stabilized lower portion 930.

According to another example embodiment, the lower portion 930 of the microneedle 900 including the 3D structure shell according to an example embodiment may include an inner pillar shell in a pyramidal shape or a cylindrical shape, and may also have a donut shape or a porous shape based on a size and a number of inner pillar shells.

The lower portion 930 is formed of a melting material that connects the base portion 10 and the microneedle 900 including the 3D structure shell and separates the microneedle 900 including the 3D structure shell from the base portion 10. For example, the lower portion 930 may be formed of a water-soluble material and may penetrate into the skin S quickly melt, thereby rapidly separating the microneedle 900 including the 3D structure shell formed on the base portion 10.

Here, similar to the upper portion 910 and the middle portion 920 that penetrate into the skin S, the lower portion 930 may be formed of a water-soluble substance. Here, the lower portion 930 may be formed of a substance that further quickly melts than those of the upper portion 910 and the middle portion 920 among water-soluble substances. The upper portion 910 is to further facilitate skin puncture, the middle portion 920 is to further efficiently administer the drug, and the lower portion 930 is to quickly separate the microneedle 900 formed on the base portion 10 and to estimate a penetration depth of the microneedle 900 into the skin S. Therefore, the microneedle 900 including the 3D structure shell according to an example embodiment includes the upper portion 910, the middle portion 920, and the lower portion 930 in a structure of three or more layers formed of different substances.

Referring to FIG. 9, the microneedle 900 including the 3D structure shell according to an example embodiment may be formed on the base portion 10. A drug is not provided to the base portion 10 and the base portion 10 is separable after the microneedle 900 including the upper portion 910, the middle portion 920, and the lower portion 930 penetrates into the skin S. For example, the base portion 10 may be provided in a patch form and may closely attach to the skin S.

Dissimilar to the microneedle 900 that penetrates into the skin S, the base portion 10 may be formed of a non-water-soluble substance that does not melt. Therefore, the base portion 10 may guide feed of a quantitative drug contained in the middle portion 920 by not interfering with the penetrating power of the microneedle 900.

For example, the base portion 10 may be formed of at least one from a group including polyethylene (PE), polypropylene (PP), polytetrafluoroethylene (PTFE), polymethyl methacrylate (PMMA), ethylene vinyl acetate (EVA), polycaprolactone (PCL), polyurethane (PU), polyethylene terephthalate (PET), polyethylene glycol (PEG), polyvinyl alcohol (PVA), polylactide (PLA), polylactide-glycolide copolymer (PLGA), and polyglycolide (PGA).

Referring to FIG. 9, the microneedle 900 including the 3D structure shell according to an example embodiment may form, in a tree-shaped structure of three or more layers, the middle portion 920 formed of a compound containing a drug component, the upper portion 910 provided at a top end of the middle portion 920 and configured to facilitate penetration into the skin S, and the lower portion 930 configured to support the middle portion 920 and to facilitate separation from the base portion 10, thereby enhancing preservation of the drug, facilitating penetration into the skin, enabling quantitative administration of the drug, and increasing a melting rate due to an increase in a surface area.

Also, since the microneedle 900 including the 3D structure shell according to an example embodiment is in a tree-shaped structure of three or more layers and thus, it is possible to increase a penetration rate (60% or more) of the structure and an absorption rate of useful components into the skin by minimizing penetration resistance caused by skin elasticity when attaching to the skin. Also, the tree-shaped microneedle 900 applies the structure of three or more layers to maximize mechanical strength of the structure and facilitating penetration into the skin.

Also, the upper portion 910 and the middle portion 920 each in a conical shape or a pyramidal shape and the lower portion 930 in a pyramidal shape or a cylindrical shape that form the microneedle 900 including the 3D structure shell according to an example embodiment are manufactured using a 3D printing technique. Since the present invention employs a 3D printing scheme, it is possible to acquire a very short attachment time, high precision, and low price compared to the conventional method. Also, it is possible to increase the number density of microneedles 900 in a micro patch and to improve an aspect ratio.

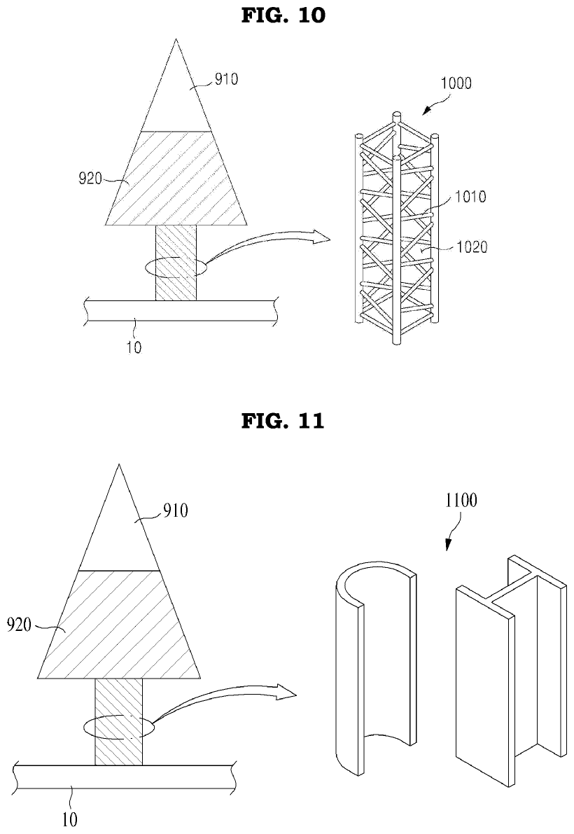

FIG. 10 illustrates a cross-sectional view of a microneedle including a 3D structure shell according to an example embodiment.

Referring to FIG. 10, the microneedle 900 including the 3D structure shell according to an example embodiment includes the lower portion 930 of the 3D structure shell, the middle portion 920, and the upper portion 910.

The lower portion 930 of the microneedle 900 including the 3D structure shell according to an example embodiment is formed as a 3D structure shell 1000 in which a plurality of units including a plurality of straight members 1020 extending in different directions are coupled.

Referring to FIG. 10, the lower portion may be the 3D structure shell 1000 in a truss structure in which a unit in which a plurality of straight members 1010 extending in different directions are arranged in a triangular shape is coupled and a plurality of units connected in the triangular shape are stacked. Also, the lower portion 930 may maintain a space 1020 between the plurality of straight members 1010 coupled as the plurality of units and in the 3D structure shell and may adjust a melting rate after penetration into the skin by adjusting the space 1020.

For example, the lower portion 930 of FIG. 10 may represent a skeleton structure constructed by arranging the plurality of straight members 1010 in a form of one or more triangles and by connecting the respective members at a node. A surface area of the lower portion 930 may increase due to the plurality of spaces 1020 formed in the skeleton structure and, due to the increased surface area, the lower portion 930 may penetrate into the skin S and a melting rate may be improved.

Further, the truss structure applied to the lower portion 930 according to an example embodiment refers to a structure form that applies to a building and is configured by connecting the plurality of straight members 1010 extending in different directions and is structurally stabilized accordingly. Therefore, the microneedle 900 including the 3D structure shell according to an example embodiment may maintain strength of the microneedle 900 using the structurally stabilized lower portion 930.

FIG. 11 illustrates a cross-sectional view of a microneedle including a 3D structure shell according to another example embodiment.

Referring to FIG. 11, the microneedle 900 including the 3D structure shell according to another example embodiment includes the lower portion 930 of the 3D structure shell, the middle portion 920, and the upper portion 910.

The lower portion 930 of the microneedle 900 including the 3D structure shell according to another example embodiment is formed as a 3D structure shell 1100 in a pillar shape with a closed curved surface having a character-shaped cross section.

Referring to FIG. 11, the lower portion 930 is the 3D structure shell 1100 that represents a pillar shape having character-shaped cross section of an approximately alphabetical character “C” or “H” or an irregular cross section and formed at the same height as that of the lower portion 930. Here, although the lower portion 930 is in a 3D structure of a pillar in a closed curved surface form in which a hollow (empty) space is maintained, the inside may be filled with the same material as that of the 3D structure shell 1100 depending on example embodiments.

The microneedle 900 including the 3D structure shell according to another example embodiment may include the lower portion 930 that is the 3D structure shell 1100 in a shape of a pillar formed as a closed curved surface such as an alphabetical character “C” or “H” and at a height of the lower portion 930 and thereby enhance a melting rate after penetration into the skin S due to an increase in a surface area caused by at least one of the form, a thickness of the pillar that forms the closed curved surface, and the height of the lower portion 930. That is, the microneedle 900 including the 3D structure shell according to another example embodiment may adjust a melting rate inside the skin S by adjusting the form and the height of the lower portion 930.

Here, although FIG. 11 illustrates a form of the lower portion 930 according to another example embodiment as an alphabetical character “C” or “H”, the microneedle 900 including the 3D structure shell according to another example embodiment is not limited thereto and may include the lower portion 930 that is structurally stable while increasing a surface area of the lower portion 930 and has a cross section in a shape of at least one of an alphabetical character, a number, a hieroglyph, a Korean alphabet, and a Roman number, which may maintain strength of the microneedle 900.

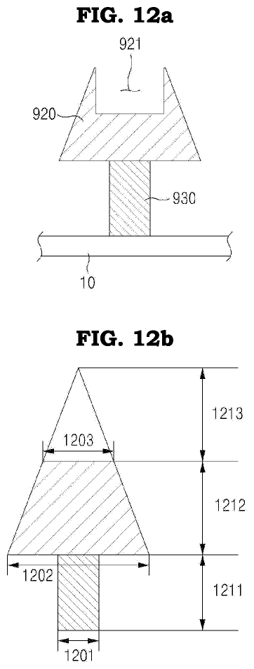

FIGS. 12A and 12B illustrate cross-sectional views to describe a structural characteristic of a microneedle according to an example embodiment.

In detail, FIG. 12A illustrates a cross-sectional view of a microneedle including a 3D structure shell according to an example embodiment and FIG. 12B illustrates a cross-sectional view of a microneedle in a structure of three or more layers according to an example embodiment.

The microneedle 900 including the 3D structure shell according to an example embodiment basically includes the middle portion 920 formed of a compound including a drug component, that is, a solidified material, and depending on example embodiments, may include the middle portion 920 in which a cavity 921 is formed to contain a drug in a liquid state.

Referring to FIG. 12A, the microneedle 900 including the 3D structure shell according to an example embodiment may include the middle portion 920 that includes the cavity 921. The cavity 921 may be formed in a groove form within the middle portion 920 and may be formed with a shape and a size for containing a drug.

Here, the drug to be contained in the cavity 921 may be a liquid drug to be injected and may be a solid drug (not shown) in a form of a capsule (micro-sphere) depending on other example embodiments. For example, the solid drug may be a polyhedron, such as a circular shape, an oval shape, a capsule type, a hexahedron, and a square prism, and may be formed using different sizes and shapes based on a type of a drug that penetrates into and acts on the skin S, strength of the drug, intensity of the drug, a state of the drug, an administration point in time of the drug, an administration time of the drug, an administration amount of the drug and a subject of the drug.

Here, a cavity surface to contact the drug may be coated with a waterproof material. When the microneedle 900 including the 3D structure shell according to an example embodiment includes the cavity 921, the microneedle 900 may contain a drug in a liquid state. Therefore, the drug may be absorbed in the middle portion 920 and thus, to prevent this, the cavity surface is coated with the waterproof material.

For example, the cavity surface may be coated with a waterproof agent that includes a mineral-based material or a lipid-based material. Here, the waterproof agent may include at least one of beeswax, oleicacid, soy fatty acid, castor oil, phosphatidylcholine, d-α-tocopherol/vitamin E, corn oil mono-ditridiglycerides, cottonseed oil, olive oil, peanut oil, peppermint oil, safflower oil, sesame oil, soybean oil, hydrogenated vegetable oils, hydrogenated soybean oil, caprylic/capric triglycerides derived from coconut oil or palm see oil, and phosphatidylcholine, or may be formed using a mixture thereof.

Depending on example embodiments, the cavity surface may be coated with a different waterproof agent based on a type and a state of a drug to be injected into the cavity 921 and the cavity 921 may be formed in the middle portion 920 to have a different size, height, and shape based on a type of the drug, a state of the drug, an administration point in time of the drug, an administration time of the drug, and an administration amount of the drug.

Referring to FIG. 12B, the microneedle 900 including the 3D structure shell according to an example embodiment is a microstructure configured in a structure of three or more layers and includes the upper portion 910 and the middle portion 920 each in a pyramidal shape or a conical shape and the lower portion 930 in a pyramidal shape or a cylindrical shape. Here, although FIGS. 12A and 12B illustrate the lower portion 930 in a pyramidal shape or a cylindrical shape in the microneedle 900 including the 3D structure shell according to an example embodiment, it may represent the 3D structure shells 1000 and 1100 of FIGS. 10 and 11.

Referring to FIG. 12B, a bottom diameter 1202 of the middle portion 920 is greater than a bottom diameter 1203 of the upper portion 910 or a bottom diameter 1201 of the lower portion 930, and the bottom diameter 1203 of the upper portion 910 is greater than the bottom diameter 1201 of the lower portion 930. Here, sizes may be determined in order of the bottom diameter 1202 of the middle portion 920, the bottom diameter 1203 of the upper portion 910, and the bottom diameter 1201 of the lower portion 930.

Also, a height 1212 of the middle portion 920 may be greater than a height 1213 of the upper portion 910 and an addition of the height 1212 of the middle portion 920 and the height 1213 of the upper portion 910 may be greater than or less than a height 1211 of the lower portion 930. That is, in the microneedle 900 including the 3D structure shell according to an example embodiment, the height 1212 of the middle portion 920 may be highest and the height 1213 of the upper portion 910 may be equal to the height 1211 of the lower portion 930, which may differ depending on example embodiments to which the microneedle 900 including the 3D structure shell according to an example embodiment applies. Here, the height 1211 of the lower portion 930, the height 1212 of the middle portion 920, and the height 1213 of the upper portion 910 in the microneedle 900 according to an example embodiment are not limited to FIG. 12B and may be variously defined depending on example embodiments.

Since the cavity 921 for containing a drug is formed in the middle portion 920 of the microneedle 900 including the 3D structure shell according to an example embodiment, the middle portion 920 may be formed with the largest volume, the largest bottom diameter 1202, and the highest height 1212. The upper portion 910 may be in a pyramidal shape or a conical shape to penetrate into the skin S and the bottom diameter 1203 of the upper portion 910 is equal to a top (or a tip) of the middle portion 920 and may be determined based on a width of a cross-sectional area of a tip of a truncated pyramid or a truncated cone that forms the middle portion 920. Also, the height 1213 of the upper portion 910 may be determined based on a shape of the truncated pyramid or the truncated cone of the middle portion 920.

The lower portion 930 of the microneedle 900 including the 3D structure shell according to an example embodiment functions to support the upper portion 910 and the middle portion 920 in the microneedle 900 and may represent a depth of penetration into the skin. Therefore, the volume and the bottom diameter 1201 of the lower portion 930 may be less than those of the upper portion 910 and the middle portion 920. Here, the height 1211 of the lower portion 930 may be determined based on the depth of penetration into the skin.

The lower portion 930 is in a shape of the 3D structure shell and includes the bottom diameter 1201 less than the bottom diameter 1203 of the upper portion 910 and the bottom diameter 1202 of the middle portion 920, and has the volume less than those of the upper portion 910 and the middle portion 920. The lower portion 930 represents a depth of the microneedle 900 that penetrates into the skin S and is configured to support the upper portion 910 and the middle portion 920 and thus, minimizes an area, volume, and weight of the microneedle 900 including the 3D structure shell according to an example embodiment. Accordingly, the lower portion 930 may support a liquid drug to be quantitatively administered due to an appropriate size, height, and diameter according to the depth of the microneedle 900 that penetrates into the skin S.

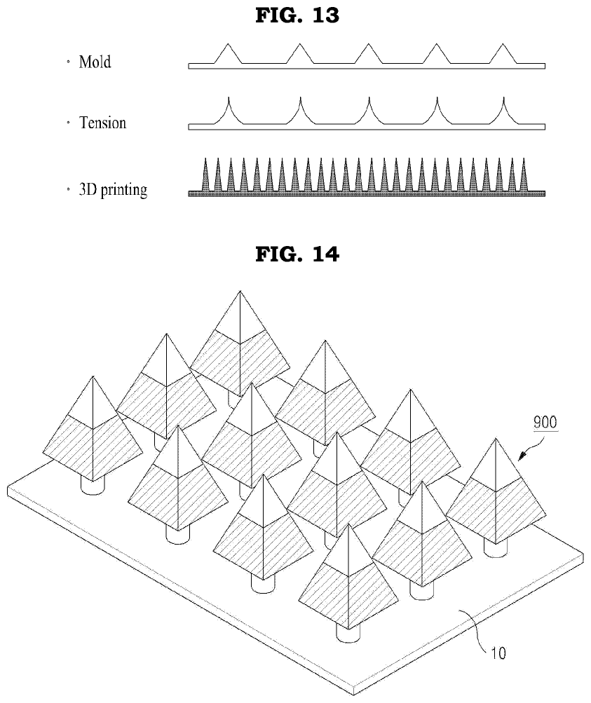

FIG. 13 illustrates an example of comparing a conventional method and a microneedle manufactured using a method according to the present invention, and FIG. 14 illustrates a perspective view of a microneedle patch manufactured according to an example embodiment.

Referring to FIG. 13, while a mold scheme and a tension scheme exhibit the low number density of microneedles, a microneedle including a 3D structure shell according to an example embodiment manufactured using a lamination scheme, for example, a 3D printing scheme exhibits the very high number density compared to the conventional method in which the number density is low due to limitations found in the mold scheme and the tension scheme. Also, the microneedle manufactured by the method according to the present invention has a higher aspect ratio compared to the mold scheme and the tension scheme. Also, the method according to the present invention may adjust the aspect ratio of the microneedle and the aspect ratio may be determined based on a field in which the microneedle of the present invention is used, for example, a field according to a treatment use and a medical use.

The method (3D printing) according to the present invention is advantageous in terms of skin puncture and painless compared to the mold scheme and has the high number density of microneedles compared to the mold scheme and the tension scheme. Also, compared to the conventional method, the method according to the present invention may acquire a very short attachment time and high precision. Since a lamination scheme, for example, a 3D printing scheme is used, manufacturing price is low and scalability is high accordingly. As described above, the method according to the present invention is very advantageous in technical and economical aspects compared to the conventional method, for example, the mold scheme and the tension scheme.

That is, since the microneedle implemented by a lamination technique with the method according to the present invention has a high aspect ratio, skin puncture is excellent, pain is very low, and the number density is high, which leads to significantly decreasing an attachment time. In addition, the present invention may implement the microneedle with a high precision of about 5 micrometers and may place a desired drug at a desired position, thereby acquiring high scalability.

Referring to FIG. 14, the microneedle 900 manufactured as described above may be provided as a microneedle patch in which a plurality of microneedles 900 are formed on the base portion 10 and may be easily applied to a medical field. That is, the present invention may secure high competitiveness in a medical market field by manufacturing the microneedle 900 in a structure of three or more layers by applying a lamination scheme using 3D printing.

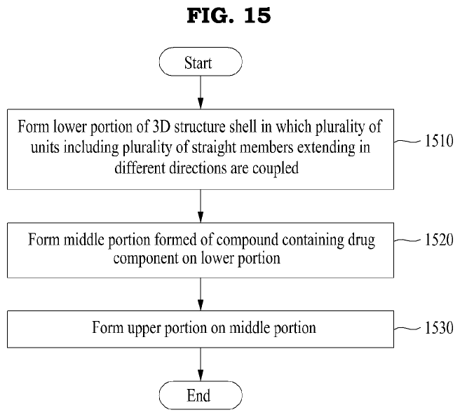

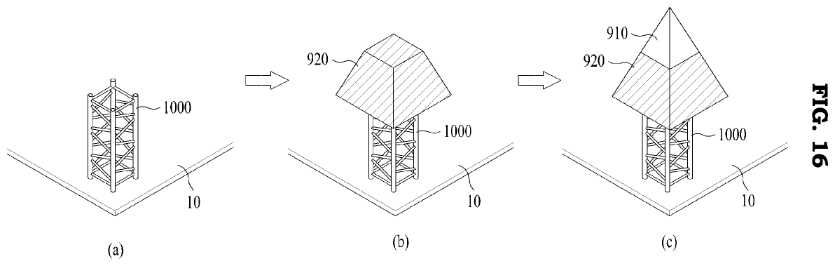

FIG. 15 illustrates a flowchart of a method of manufacturing a microneedle including a 3D structure shell according to an example embodiment, and FIG. 16 illustrates a process of manufacturing a microneedle using a method of manufacturing a microneedle including a 3D structure shell according to an example embodiment.

The microneedle 900 of FIG. 16 manufactured through the manufacturing method of FIG. 15 may be manufactured through a 3D printing scheme.

Referring to FIG. 15 and (a) of FIG. 16, in operation 1510, a lower portion 1000 that represents a 3D structure shell in which a plurality of units including a plurality of straight members extending in different directions are coupled is formed. The method of manufacturing the microneedle including the 3D structure shell according to an example embodiment may form, on the base portion 10, the lower portion 1000 that is the 3D structure shell in a truss structure in which a unit in which a plurality of straight members extending in different directions are arranged in a triangular shape is coupled and a plurality of units connected in the triangular shape are stacked.

The lower portion 1000 may have a predetermined diameter and height, and may represent a depth of the microneedle 900 that penetrates into the skin S. For example, a penetration depth of the upper portion 910 and the middle portion 920 containing a drug into the skin S may be estimated based on the diameter and the height of the lower portion 1000, and the height of the lower portion 1000 according to a depth of the drug to be penetrated and a space between the plurality of straight members coupled as the plurality of units and inside the 3D structure shell 1000 may be adjusted based on a type of the drug, a state of the drug, an administration point in time of the drug, an administration time of the drug, and an administration amount of the drug.

The lower portion 1000 may represent a skeleton structure constructed by arranging the plurality of straight members in a form of one or more triangles and by connecting the respective members at a node. A surface area of the lower portion 1000 may increase due to a plurality of spaces formed in the skeleton structure. Therefore, due to the increased surface area, the lower portion 1000 may penetrate into the skin S and a melting rate may be improved.

Further, the truss structure applied to the lower portion 1000 according to an example embodiment refers to a structure form that applies to a building and is configured by connecting the plurality of straight members extending in different directions and is structurally stabilized accordingly. Therefore, the microneedle 900 including the 3D structure shell according to an example embodiment may maintain strength of the microneedle 900 using the structurally stabilized lower portion 1000.

The lower portion 1000 is formed of a melting material that connects the base portion 10 and the microneedle 900, and separates the microneedle 900 from the base portion 10. For example, the lower portion 1000 may be formed of a water-soluble substance and may penetrate into the skin S and quickly melt, thereby rapidly separating the microneedle 900 formed on the base portion 10.

Here, similar to the upper portion 910 and the middle portion 920 that penetrate into the skin S, the lower portion 1000 may be formed of a water-soluble substance. Here, the lower portion 1000 may be formed of a substance that further quickly melts than those of the upper portion 910 and the middle portion 920 among water-soluble substances. Here, the upper portion 910 is to further facilitate skin puncture, the middle portion 920 is to further efficiently administer the drug, and the lower portion 1000 is to quickly separate the microneedle 900 formed on the base portion 10 and to estimate a penetration depth of the microneedle 900 into the skin S. Therefore, the microneedle 900 according to an example embodiment includes the upper portion 910, the middle portion 920, and the lower portion 1000 in a structure of three or more layers formed of different substances.

In operation 1520, the middle portion 920 that penetrates into the skin and formed of a compound containing a drug component is formed on the lower portion 1000. Referring to (b) of FIG. 16, the method of manufacturing the microneedle including the 3D structure shell according to an example embodiment may form, on the lower portion 1000, the solidified middle portion 920 formed of a compound containing a drug component. Here, although (b) of FIG. 16 illustrates the middle portion 920 formed of the compound containing the drug component, the middle portion 920 of the microneedle 900 according to an example embodiment may include a cavity capable of containing a drug in a liquid state.

According to an example embodiment, the middle portion 920 may be formed of a water-soluble substance, which is similar to the upper portion 910 that penetrates into the skin S. Here, since the middle portion 920 is formed of the compound containing the drug component, the middle portion 920 may be formed of a substance different from those of the upper portion 910 and the lower portion 1000.

In operation 1530, the upper portion 910 is formed on the middle portion 920. Referring to (c) of FIG. 16, the method of manufacturing the microneedle including the 3D structure shell according to an example embodiment may form the upper portion 910 provided at a top end of the middle portion 920 and configured to facilitate penetration into the skin S. The upper portion 910 may have a sharp tip shape based on a penetration direction for penetrating into the skin S. For example, the upper portion 910 may be formed in a pyramidal shape or a conical shape such as a triangular shape, a quadrangular shape, a pentagonal shape, and a hexagonal shape, thereby facilitating penetration into the skin S.

Each of the upper portion 910, the middle portion 920, and the lower portion 1000 of the microneedle 900 including the 3D structure shell according to an example embodiment may be formed of a different material. Although the upper portion 910, the middle portion 920, and the lower portion 1000 may be formed of the same water-soluble substance, the upper portion 910 configured to facilitate penetration, the middle portion 920 formed of a compound containing a drug component, and the lower portion 1000 configured to support the middle portion 920 and to facilitate separation from the base portion 10 may be formed of different types of substances among water-soluble substances.

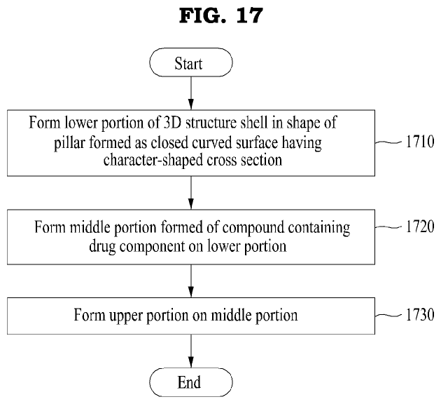

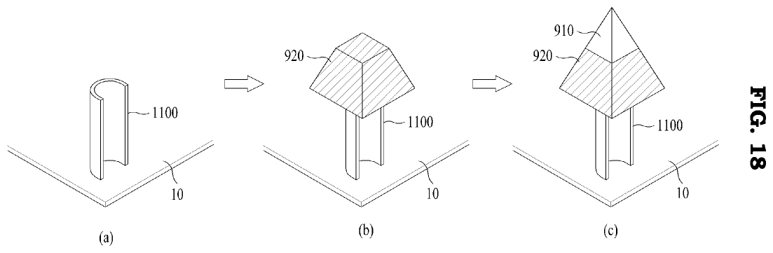

FIG. 17 illustrates a flowchart of a method of manufacturing a microneedle including a 3D structure shell according to another example embodiment, and FIG. 18 illustrates a process of manufacturing a microneedle using a method of manufacturing a microneedle including a 3D structure shell according to an example embodiment.

The microneedle 900 of FIG. 18 manufactured through the manufacturing method of FIG. 17 may be manufactured through a 3D printing scheme.

Referring to FIG. 17 and (a) of FIG. 18, in operation 1710, a lower portion 1100 that represents a 3D structure shell in a pillar shape with a closed curved surface having a character-shaped cross section. The method of manufacturing the microneedle including the 3D structure shell according to another example embodiment may form, on the base portion 10, the lower portion 1100 that is the 3D structure shell representing a pillar shape having a character-shaped cross section of an alphabetical character “C” or “H” or an irregular cross section and formed at the same height as that of the lower portion 1100.

The lower portion 1100 may have a predetermined diameter and height, and may represent a depth of the microneedle 900 that penetrates into the skin S. For example, a penetration depth of the upper portion 910 and the middle portion 920 containing a drug into the skin S may be estimated based on the diameter and the height of the lower portion 1100. The microneedle 900 including the 3D structure shell according to another example embodiment may include the lower portion 1100 that is the 3D structure shell in a shape of a pillar formed as a close curved surface such as an alphabetical character “C” or “H” and at a height of the lower portion 1100 according to a depth of a drug to be penetrated based on a type of the drug, a state of the drug, an administration point in time of the drug, an administration time of the drug, and an administration amount of the drug and thereby enhance a melting rate after penetration into the skin S due to an increase in a surface area caused by at least one of the form, a thickness of the pillar that forms the closed curved surface, and the height of the lower portion 1100. That is, the microneedle 900 including the 3D structure shell according to another example embodiment may adjust a melting rate inside the skin S by adjusting the form, the thickness, and the height of the lower portion 1100.

Here, although FIG. 18 illustrates a form of the lower portion 1100 according to another example embodiment as an alphabetical character “C”, the microneedle 900 including the 3D structure shell according to another example embodiment is not limited thereto and may include the lower portion 1100 that is structurally stable while increasing a surface area of the lower portion 1100 and has a cross section in a shape of at least one of an alphabetical character, a number, a hieroglyph, Korean alphabet, and a Roman number, which may maintain strength of the microneedle 900.

The lower portion 1100 is formed of a melting material that connects the base portion 10 and the microneedle 900, and separates the microneedle 900 from the base portion 10. For example, the lower portion 1100 may be formed of a water-soluble substance and may penetrate into the skin S and quickly melt, thereby rapidly separating the microneedle 900 formed on the base portion 10.

Here, similar to the upper portion 910 and the middle portion 920 that penetrate into the skin S, the lower portion 1100 may be formed of a water-soluble substance. Here, the lower portion 1100 may be formed of a substance that further quickly melts than those of the upper portion 910 and the middle portion 920 among water-soluble substances. The upper portion 910 is to further facilitate skin puncture, the middle portion 920 is to further efficiently administer the drug, and the lower portion 1100 is to quickly separate the microneedle 900 formed on the base portion 10 and to estimate a penetration depth of the microneedle 900 into the skin S. Therefore, the microneedle 900 according to another example embodiment includes the upper portion 910, the middle portion 920, and the lower portion 1100 in a structure of three or more layers formed of different substances.

In operation 1720, the middle portion 920 that penetrates into the skin and formed of a compound containing a drug component is formed on the lower portion 1100. Referring to (b) of FIG. 18, the method of manufacturing the microneedle including the 3D structure shell according to an example embodiment may form, on the lower portion 1100, the solidified middle portion 920 formed of a compound containing a drug component. Here, although (b) of FIG. 18 illustrates the middle portion 920 formed of the compound containing the drug component, the middle portion 920 of the microneedle 900 according to another example embodiment may include a cavity capable of containing a drug in a liquid state.

According to another example embodiment, the middle portion 920 may be formed of a water-soluble substance, which is similar to the upper portion 910 that penetrates into the skin S. Here, since the middle portion 920 is formed of the compound containing the drug component, the middle portion 920 may be formed of a substance different from those of the upper portion 910 and the lower portion 1100.

In operation 1730, the upper portion 910 is formed on the middle portion 920. Referring to (c) of FIG. 18, the method of manufacturing the microneedle including the 3D structure shell according to another example embodiment may form the upper portion 910 provided at a top end of the middle portion 920 and configured to facilitate penetration into the skin S. The upper portion 910 may have a sharp tip shape based on a penetration direction for penetrating into the skin S. For example, the upper portion 910 may be formed in a pyramidal shape or a conical shape such as a triangular shape, a quadrangular shape, a pentagonal shape, and a hexagonal shape, thereby facilitating penetration into the skin S.

Each of the upper portion 910, the middle portion 920, and the lower portion 1100 of the microneedle 900 including the 3D structure shell according to another example embodiment may be formed of a different material. Although the upper portion 910, the middle portion 920, and the lower portion 1100 may be formed of the same water-soluble substance, the upper portion 910 configured to facilitate penetration, the middle portion 920 formed of a compound containing a drug component, and the lower portion 1100 configured to support the middle portion 920 and to facilitate separation from the base portion 10 may be formed of different types of substances among water-soluble substances.

The example embodiments are to enhance preservation of a drug and to allow a solid drug in a structure in which a drug is contained to penetrate into the skin by manufacturing a microneedle containing the solid drug in a structure of three or more layers including a middle portion containing the solid drug, an upper portion provided at a top end of the middle portion and configured to facilitate penetration into the skin, and a lower portion configured to support the middle portion. Here, the microneedle containing the solid drug according to an example embodiment is in the structure of three or more layers.

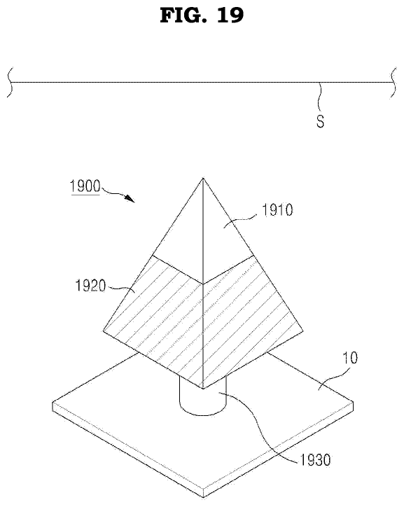

Hereinafter, example embodiments are described with reference to FIGS. 19 to 26.

FIG. 19 illustrates a perspective view of a microneedle containing a solid drug according to an example embodiment.

Referring to FIG. 19, a microneedle 1900 containing a solid drug according to an example embodiment includes an upper portion 1910, a middle portion 1920, and a lower portion 1930.

The upper portion 1910 is provided at a top end of the middle portion 1920 and facilitates penetration into the skin S. The upper portion 1910 may have a sharp tip shape based on a penetration direction for penetrating into the skin S. For example, the upper portion 1910 may be formed in a pyramidal shape or a conical shape such as a triangular shape, a quadrangular shape, a pentagonal shape, and a hexagonal shape, thereby facilitating penetration into the skin S. Here, to facilitate puncture of the skin S, the upper portion 1910 may be formed of a material with stronger strength than those of the middle portion 1920 and the lower portion 1930.

The upper portion 1910 according to an example embodiment allows the microneedle 1900 containing a solid drug to easily penetrate into the skin S and may protect the middle portion 1920 containing the solid drug.

According to an example embodiment, the upper portion 1910 may be formed of a water-soluble substance that penetrates into the skin S and melts. For example, the water-soluble substance may include at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropyl methylcellulose (HPMC), ethylcellulose (EC), hydroxypropylcellulose (HPC), carboxymethylcellulose, cyclodextrin, and gentiobiose.

The middle portion 1920 is capable of penetrating into the skin S through the upper portion 1910 and contains the solid drug in the cavity. When the middle portion 1920 penetrates into the skin S through the upper portion 1910, the solid drug contained in the cavity may be absorbed into the skin S. Here, the solid drug is in a structure in which a drug containing a drug is contained and a size and a type of the drug to be contained in the structure in which the drug is contained may be adjusted based on a degree of the drug that penetrates into the skin S, performance, a state of a subject (or a user) to which the drug applies, and a melting time.

The middle portion 1920 is in a truncated pyramidal shape or a truncated conical shape, such as a triangle, a square, a pentagon, and a hexagon from which the upper portion 1910 is removed, and includes a cavity area in which the solid drug is containable. Here, the cavity area may be provided in an upper area above the center of the middle portion 1920. However, depending on example embodiments, a position, a size, and a shape of the cavity area may be variously applied based on an administration point in time, an administration time, and an administration amount of the solid drug. Further, a size and a position of the cavity may be adjusted based on an amount, an evaporation rate, and a temperature of the sold drug, a shape of the middle portion 1920 for manufacture of the microneedle 900, viscosity of the drug, concentration of the drug, a solvent used, and a thickness that covers a top of the cavity.

Similar to the upper portion 1910 that penetrates into the skin S, the middle portion 1920 may be formed of a water-soluble substance. Here, the middle portion 1920 includes the cavity and the solid drug contained in the cavity and thus, may be formed of a substance different from that of the upper portion 1910 among water-soluble substances.

Further, when a solid material contained in the cavity area is included in the middle portion 1920, a portion of the solid material may be absorbed into a material of the middle portion 1920. Therefore, the middle portion 1920 may be formed of a water-soluble substance different from that of the upper portion 1910 and the surface of the cavity in which the solid material is to be contained may be coated with a waterproof material.

The solid drug contained in the cavity included in the middle portion 1920 may be formed of a biocompatible material and an additive. For example, the biocompatible material may include at least one of carboxymethylcellulose (CMC), hyaluronic acid (HA), alginic acid, pectin, carrageenan, chondroitin sulfate, dextran sulfate, chitosan, polylysine, carboxymethyl chitin, fibrin, agarose, pullulan, polyanhydride, polyorthoester, polyetherester, polyesteramide, poly butyric acid, poly valeric acid, polyacrylate, ethylene-vinyl acetate polymer, acrylic-substituted cellulose acetate, polyvinyl chloride, polyvinyl fluoride, polyvinyl imidazole, chlorosulphonate polyolefins, polyethylene oxide, polyvinylpyrrolidone (PVP), hydroxypropyl methylcellulose (HPMC), ethylcellulose (EC), hydroxypropylcellulose (HPC), carboxymethyl cellulose, cyclodextrin, maltose, lactose, trehalose, cellobiose, isomaltose, turanose, and lactulose, or may include at least one of a copolymer of monomers that form such a polymer and cellulose.

Also, the additive may include at least one of trehalose, oligosaccharide, sucrose, maltose, lactose, cellobiose, hyaluronic acid, alginic acid, pectin, carrageenan, chondroitin sulfate, dextran sulfate, chitosan, polylysine, collagen, gelatin, carboxymethyl chitin, fibrin, agarose, polyvinylpyrrolidone (PVP), polyethylene glycol (PEG), polymethacrylate, hydroxypropyl methylcellulose (HPMC), ethylcellulose (EC), hydroxypropylcellulose (HPC), carboxymethyl cellulose, cyclodextrin, gentiobiose, alkyltrimethylammonium bromide (cetrimide), hexadecyltrimethylammonium bromide (CTAB), gentian violet, benzethonium chloride, docusate sodium salta SPAN-type surfactant, polysorbate (Tween), sodium dodecyl sulfate (SDS), benzalkonium chloride, and glyceryl oleate.

Also, the solid drug contained in the cavity of the middle portion 1920 may be formed by mixing a biocompatible material and an active ingredient. The active ingredient includes protein/peptide medicine. However, it is provided as an example only and the active ingredient includes at least one of hormone, hormone analog, enzyme, enzyme inhibitor, signal transduction protein or a portion thereof, antibody or a portion thereof, single-chain antibody, binding protein or a binding domain thereof, antigen, adhesion protection, structural protein, regulatory protein, toxin protein, cytokine, a transcription factor, a blood coagulation factor, and vaccine. In detail, the protein/peptide medicine may include one of insulin, insulin-like growth factor 1 (IGF-1), growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), TNF (tumor necrosis factor), atobisban, buserelin, cetrorelix, deslorelin, desmopressin, dynorphin A 1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormonereleasing hormone (LHRH), nafarelin, parathormone, pramlintide, T-20 (enfuvirtide), thymalfasin, and ziconotide.

Also, a solvent of the drug component contained in the cavity of the middle portion 1920 may dissolve the biocompatible material. The solvent may include at least one of inorganic and organic solvents that include DI water, methanol, ethanol, chloroform, dibutyl phthalate, dimethyl phthalate, ethyl lactate, glycerin, isopropyl alcohol, lactic acid, propylene glycol, and the like.

The microneedle 1900 containing the solid drug according to an example embodiment may administer a quantitative drug by forming a cavity of a specific area in the middle portion 1920 and by containing, in the cavity, a solid drug in a structure in which a drug containing a drug is contained to penetrate into the skin S. Accordingly, the present invention may enhance preservation of the drug, may facilitate penetration of the solid drug into the skin, and may administer, into the skin S, the solid drug in the structure in which the drug is contained. Further, the present invention may include, in the cavity, a plurality of solid drugs containing different drugs and thereby administer, into the skin S at a time, the solid drugs that provide different actions and effects.

The lower portion 1930 supports the middle portion 1920. The lower portion 1930 is in a pyramidal shape or a cylindrical shape, such as a triangle, a square, a pentagon, and a hexagon, and supports the upper portion 1910 and the middle portion 1920.

The lower portion 1930 has a predetermined diameter and height, and may represent a depth of the microneedle 1900 containing the solid drug that penetrates into the skin S. For example, a penetration depth of the upper portion 1910 and the middle portion 1920 containing the solid drug into the skin S may be estimated based on the diameter and the height of the lower portion 1930. The height of the lower portion 1930 may be adjusted based on a type of the drug, a state of the drug, an administration point in time of the drug, an administration time of the drug, and an administration amount of the drug. Also, a diameter of the lower portion 1930 may be adjusted based on a weight and a size of the upper portion 1910 and the middle portion 1920, a level of sustaining the solid drug, and an amount of time used for the lower portion 1930 to melt in the skin S.

The lower portion 1930 is formed of a melting material that connects the base portion 10 and the microneedle 1900 containing the solid drug, and separates the microneedle 1900 containing the solid drug from the base portion 10. For example, the lower portion 1930 is formed of a water-soluble substance and thus may quickly melt, thereby rapidly separating the microneedle 1900 formed on the base portion 10.

Here, similar to the upper portion 1910 and the middle portion 1920 that penetrate into the skin S, the lower portion 1930 may be formed of a water-soluble substance. Here, the lower portion 1930 may be formed of a substance that further quickly melts than those of the upper portion 1910 and the middle portion 1920 among water-soluble substances. The upper portion 1910 is to further facilitate skin puncture, the middle portion 1920 is to deliver the solid drug and to further efficiently administer the solid drug, and the lower portion 1930 is to quickly separate the microneedle 1900 formed on the base portion 10 and to estimate a penetration depth of the microneedle 1900 into the skin S. Therefore, the microneedle 1900 containing the solid drug according to an example embodiment includes the upper portion 1910, the middle portion 1920, and the lower portion 1930 in a structure of three or more layers formed of different substances.

The lower portion 1930 according to an example embodiment may represent a depth of penetration into the skin as a role of supporting the upper portion 1910 and the middle portion 1920 in the microneedle 1900. Referring to FIG. 19, the lower portion 1930 is in a pyramidal shape or a cylindrical shape and has a small size and volume compared to those of the upper portion 1910 and the middle portion 1920. Accordingly, the lower portion 1930 may minimize an area, volume, and weight of the microneedle 1900 and may support a quantitative drug to be administered due to a shape of an appropriate size, height, and diameter according to a depth of the microneedle 1900 that penetrates into the skin S.

Depending on example embodiments, the lower portion 1930 of the microneedle 1900 containing the solid drug according to an example embodiment may be formed using a 3D structure shell that is in a structure in which a plurality of straight members extending in different directions are coupled or in a structure of a pillar shape formed as a closed curved surface having a character-shaped cross section.

Depending on other example embodiments, the lower portion 1930 of the microneedle 1900 containing the solid drug according to an example embodiment may include an inner pillar shell in a pyramidal shape or a cylindrical shape, and may have a donut shape or a porous shape based on a size and a number of inner pillar shells.

Referring to FIG. 19, the microneedle 1900 containing the solid drug may be formed on the base portion 10. A drug is not provided to the base portion 10 and the base portion 10 is separable after the microneedle 1900 including the upper portion 1910, the middle portion 1920, and the lower portion 1930 penetrates into the skin S. For example, the base portion 10 may be provided in a patch form and may closely attach to the skin S.

Dissimilar to the microneedle 100 that penetrates into the skin S, the base portion 10 may be formed of a non-water-soluble substance that does not melt. Therefore, the base portion 10 may guide feed of a quantitative drug contained in the middle portion 1920 by not interfering with the penetrating power of the microneedle 1900.

For example, the base portion 10 may be formed of at least one from a group including polyethylene (PE), polypropylene (PP), polytetrafluoroethylene (PTFE), polymethyl methacrylate (PMMA), ethylene vinyl acetate (EVA), polycaprolactone (PCL), polyurethane (PU), polyethylene terephthalate (PET), polyethylene glycol (PEG), polyvinyl alcohol (PVA), polylactide (PLA), polylactide-glycolide copolymer (PLGA), and polyglycolide (PGA).

Referring to FIG. 19, the microneedle 1900 containing the solid drug according to an example embodiment may form, in a tree-shaped structure of three or more layers, the middle portion 1920 containing the solid drug, the upper portion 1910 provided at a top end of the middle portion 1920 and configured to facilitate penetration into the skin S, and the lower portion 1930 configured to support the middle portion 1920 and to facilitate separation from the base portion 10, thereby enhancing preservation of the drug and enabling the solid drug in a structure in which the drug is contained to penetrate into the skin S.

Also, since the microneedle 1900 containing the solid drug according to an example embodiment is in a tree-shaped structure of three or more layers and thus, it is possible to increase a penetration rate (60% or more) of the structure and an absorption rate of useful components into the skin by minimizing penetration resistance caused by skin elasticity when attaching to the skin. Also, the tree-shaped microneedle 1900 applies a structure of three or more layers to maximize mechanical strength of the structure and facilitating penetration into the skin.

Also, the upper portion 1910 and the middle portion 1920 each in a conical shape or a pyramidal shape and the lower portion 1930 in a pyramidal shape or a cylindrical shape that form the microneedle 1900 containing the solid drug according to an example embodiment are manufactured using a 3D printing technique. Since the present invention employs a 3D printing scheme, it is possible to acquire a very short attachment time, high precision, and low price compared to the conventional method. Also, it is possible to increase the number density of the microneedles 1900 in a micro patch and improve an aspect ratio.

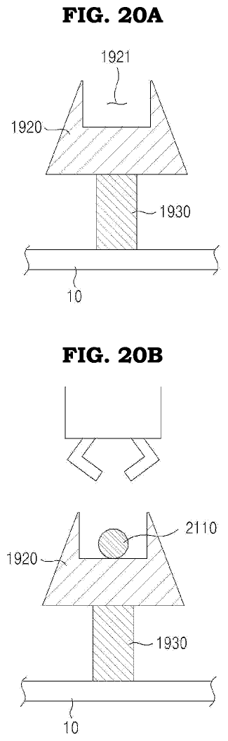

FIGS. 20A and 20B illustrate cross-sectional views of a microneedle containing a solid drug according to an example embodiment.

Referring to FIG. 20A, the microneedle 1900 containing the solid drug according to an example embodiment includes the middle portion 1920 that includes the cavity 1921. The cavity 1921 may be formed in a groove form within the middle portion 1920 and may be formed with a shape and a size for containing a solid drug.

Referring to FIGS. 20A and 20B, the microneedle 1900 containing the solid drug according to an example embodiment may include the cavity 1921 that contains a solid drug 2110. Here, it can be known that the cavity 1921 containing the solid drug 2110 is completely present within the middle portion 1920. When the solid drug 2110 is injected into an area of the cavity 1921, it is possible to seal the solid drug 2110 by blocking a cavity top. Subsequently, the microneedle 1900 containing the solid drug according to an example embodiment may be manufactured by forming the upper portion 1910 on the middle portion 1920 in which the cavity top is blocked.

Referring to FIG. 20B, the solid drug 2110 may be in a structure form in which a drug containing a drug is contained and may be positioned in an area of the cavity 1921 of the middle portion 1920 through a movement method. For example, the solid drug 2110 may be in a structure form in which a drug is contained, for example, a polyhedron such as a circular shape, an oval shape, a capsule type, a hexahedron, and a square prism, and may be formed using different sizes and shapes based on a type of a drug that penetrates into and acts on the skin S, a state of the drug, an administration point in time of the drug, an administration time of the drug, and an administration amount of the drug.

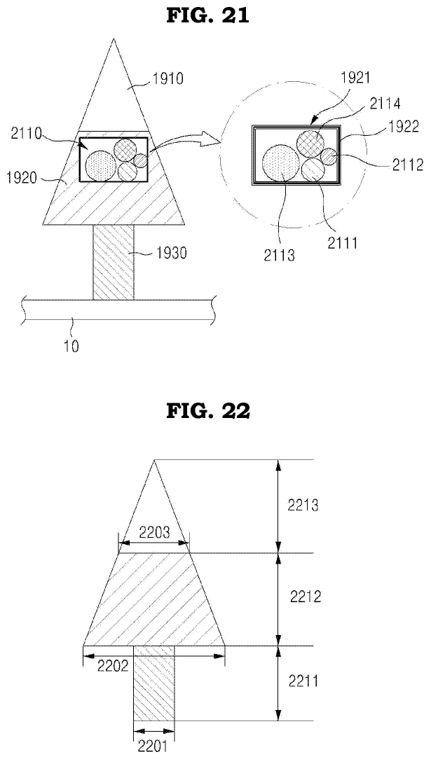

FIG. 21 illustrates a cross-sectional view of a microneedle containing a plurality of solid drugs according to an example embodiment.

Referring to FIG. 21, the microneedle 1900 containing a solid drug according to an example embodiment may include a plurality of solid drugs 2110 containing different drugs in an area of the cavity 1921.

The microneedle 1900 containing the solid drug according to an example embodiment may administer, into the skin S at a time, the solid drugs 2110 that provides different actions and effects by including, in the cavity 1921, the plurality of solid drugs 2110 containing different drugs in the cavity 1921 based on a subject to which a drug is applied after penetration into the skin S. For example, the microneedle 1900 containing the solid drug according to an example embodiment may include, in the cavity 1921 at a time, a solid drug 2111 containing a drug A, a solid drug 2112 containing a drug B, a solid drug 2113 containing a drug C, and a solid drug 2114 containing a drug D. The microneedle 1900 containing the same according to an example embodiment may penetrate into the skin S and provide different actions and effects.

Here, referring to FIG. 21, the plurality of solid drugs 2110 may be in a structure form in which a drug is contained, for example, a polyhedron such as a circular shape, an oval shape, a capsule type, a hexahedron, and a square prism, and may be formed using different sizes and shapes based on a type of a drug that penetrates into and acts on the skin S, strength of the drug, intensity of the drug, a state of the drug, an administration point in time of the drug, an administration time of the drug, an administration amount of the drug, and a subject of the drug.

Also, referring to FIG. 21, a cavity surface 1922 in contact with the solid drug 2110 may be coated with a waterproof material. The solid drug 2110 may be absorbed through the surface of the middle portion 1920. Therefore, to prevent this, the cavity surface 1922 may be coated with a waterproof material.

For example, the cavity surface 1922 may be coated with a waterproof agent that includes a mineral-based material or a lipid-based material. Here, the waterproof agent may include at least one of beeswax, oleicacid, soy fatty acid, castor oil, phosphatidylcholine, d-α-tocopherol/vitamin E, corn oil mono-ditridiglycerides, cottonseed oil, olive oil, peanut oil, peppermint oil, safflower oil, sesame oil, soybean oil, hydrogenated vegetable oils, hydrogenated soybean oil, caprylic/capric triglycerides derived from coconut oil or palm see oil, and phosphatidylcholine, or may be formed using a mixture thereof.

Depending on example embodiments, the cavity surface 1922 may be coated with a different waterproof agent based on a type and a state of the solid drug 2110 to be injected into the cavity 1921 and the cavity 1921 may be formed in the middle portion 1920 to have a different size, height, and shape based on a type of the solid drug 2110, a state of the solid drug 2110, an administration point in time of the solid drug 2110, an administration time of the solid drug 2110, and an administration amount of the solid drug 2110.

FIG. 22 illustrates a cross-sectional view of a microneedle in a structure of three or more layers containing a solid drug according to an example embodiment.

Referring to FIG. 22, the microneedle 1900 containing the solid drug according to an example embodiment is a microstructure in a structure of three or more layers and includes the upper portion 1910 and the middle portion 1920 each in a pyramidal shape or a conical shape and the lower portion 1930 in a pyramidal shape or a cylindrical shape.

Referring to FIG. 22, a bottom diameter 2202 of the middle portion 1920 is greater than a bottom diameter 2203 of the upper portion 1910 or a bottom diameter of the lower portion 1930 and the bottom diameter 2203 of the upper portion 1910 is greater than the bottom diameter 2201 of the lower portion 1930. Here, sizes may be determined in order of the bottom diameter 2202 of the middle portion 1920, the diameter 2203 of the upper portion 1910, and the bottom diameter 2201 of the lower portion 1930.

Also, a height 2212 of the middle portion 1920 is greater than a height 2213 of the upper portion 1910 and an addition of the height 2212 of the middle portion 1920 and the height 2213 of the upper portion 1910 may be greater than or less than a height 2211 of the lower portion 1930. That is, in the microneedle 1900 containing the solid drug according to an example embodiment, the height 2212 of the middle portion 1920 may be highest and the height 2213 of the upper portion 1910 may be equal to the height 2211 of the lower portion 1930, which may differ depending on example embodiments to which the microneedle 1900 containing the solid drug according to an example embodiment applies. Here, the height 2211 of the lower portion 1930, the height 2212 of the middle portion 1920, and the height 2213 of the upper portion in the microneedle 1900 containing the solid drug according to an example embodiment are not limited to FIG. 22 and may be variously defined depending on example embodiments.

Since the cavity 1921 for containing a solid drug is formed in the middle portion 1920 of the microneedle 1900 containing the solid drug according to an example embodiment, the middle portion 1920 may be formed with the largest volume, the largest bottom diameter 2202, and the highest height 2212. The upper portion 1910 may be in a pyramidal shape or a conical shape to penetrate into the skin S and the bottom diameter 2203 of the upper portion 1910 is equal to a top (or a tip) of the middle portion 1920 and may be determined based on a width of a cross-sectional area of a tip of a truncated pyramid or a truncated cone that forms the middle portion 1920. Also, the height 2213 of the upper portion 1910 may be determined based on a shape of the truncated pyramid or the truncated cone of the middle portion 1920.

The lower portion 1930 of the microneedle 1900 containing the solid drug according to an example embodiment functions to support the upper portion 1910 and the middle portion 1920 in the microneedle 1900 and may represent a depth of penetration into the skin. Therefore, the volume and the bottom diameter 2201 of the lower portion 1930 may be less than those of the upper portion 1910 and the middle portion 1920. Here, the height 2211 of the lower portion 1930 may be determined based on the depth of penetration into the skin.

The lower portion 1930 is in a pyramidal shape or a cylindrical shape and includes the bottom diameter 2201 less than the bottom diameter 2203 of the upper portion 1910 and the bottom diameter 2202 of the middle portion 1920, and has the volume less than those of the upper portion 1910 and the middle portion 1920. The lower portion 1930 represents a depth of the microneedle 1900 that penetrates into the skin S and is configured to support the upper portion 1910 and the middle portion 1920 and thus, minimizes an area, volume, and weight of the microneedle 1900 containing the solid drug according to an example embodiment. Accordingly, the lower portion 1930 may support a liquid drug to be quantitatively administered due to an appropriate size, height, and diameter according to the depth of the microneedle 1900 that penetrates into the skin S.

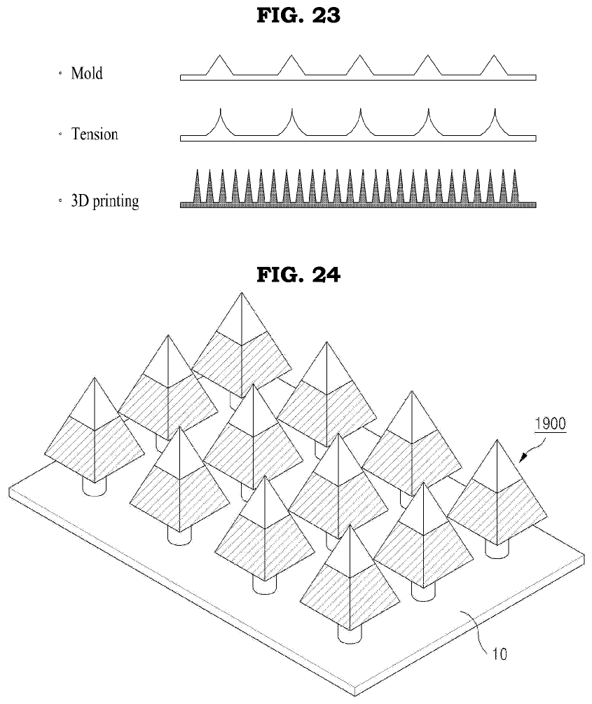

FIG. 23 illustrates an example of comparing a conventional method and a microneedle manufactured using a method according to the present invention, and FIG. 24 illustrates a perspective view of a microneedle patch manufactured according to an example embodiment.

Referring to FIG. 23, while a mold scheme and a tension scheme having the low number density of microneedles, a microneedle containing a solid drug according to an example embodiment using a lamination scheme, for example, a 3D printing scheme exhibits the very high number density compared to the conventional method due to limitations found in the mold scheme and the tension scheme. Also, the microneedle manufactured by the method according to the present invention has a higher aspect ratio compared to the mold scheme and the tension scheme. Also, the method according to the present invention may adjust the aspect ratio of the microneedle and the aspect ratio may be determined based on a field in which the microneedle of the present invention is used, for example, a field according to treatment use and medical use.

The method (3D printing) according to the present invention is advantageous in terms of skin puncture and painless compared to the mold scheme and has the high number density of microneedles compared to the mold scheme and the tension scheme. Also, compared to the conventional method, the method according to the present invention may acquire a very short attachment time and high precision. Since a lamination scheme, for example, a 3D printing scheme is used, manufacturing price is low and scalability is high accordingly. As described above, the method according to the present invention is very advantageous in technical and economical aspects compared to the conventional method, for example, the mold scheme and the tension scheme.

That is, since the microneedle implemented by a lamination technique with the method according to the present invention has a high aspect ratio, skin puncture is excellent, pain is very low, and the number density is high, which leads to significantly decreasing an attachment time. In addition, the present invention may implement the microneedle with high precision of about 5 micrometers and may place a desired drug at a desired position, thereby acquiring a high scalability.

Referring to FIG. 24, the microneedle 1900 manufactured as described above may be provided as a microneedle patch in which a plurality of microneedles 1900 are formed on the base portion 10 and may be easily applied to a medical field. That is, the present invention may secure high competitiveness in a medical market field by manufacturing the microneedle 1900 in a structure of three or more layers by applying a lamination scheme using 3D printing.

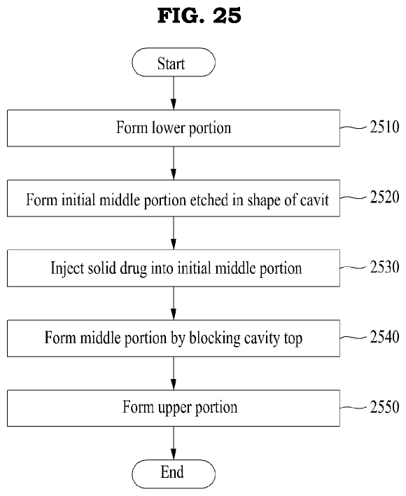

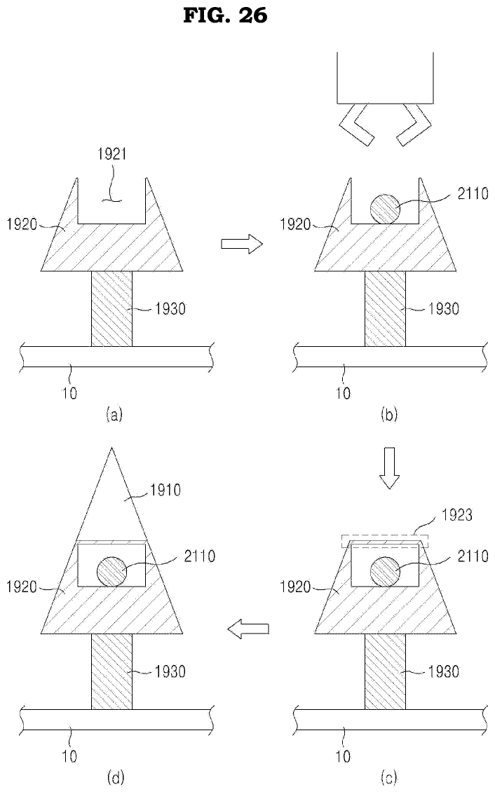

FIG. 25 illustrates a flowchart of a method of manufacturing a microneedle containing a solid drug according to an example embodiment, and FIG. 26 illustrates a process of manufacturing a microneedle containing a solid drug using a method of manufacturing the microneedle containing the solid drug according to an example embodiment.

The microneedle 1900 containing the solid drug of FIG. 26 manufactured through the manufacturing method of FIG. 25 may be manufactured using a 3D printing scheme.

Referring to FIG. 25, in operation 2510, the lower portion 1930 is formed. The method of manufacturing the microneedle containing the solid drug according to an example embodiment may form the lower portion 1930 in a pyramidal shape or a cylindrical shape on the base portion 10.

The lower portion 1930 may have a predetermined diameter and height, and may represent a depth of the microneedle 1900 that penetrates into the skin S. For example, a penetration depth of the upper portion 1910 and the middle portion 1920 containing a solid drug into the skin S may be estimated based on the diameter and the height of the lower portion 1930, and the height of the lower portion 1930 according to a depth of a drug to be penetrated may be adjusted based on a type of the drug, a state of the drug, an administration point in time of the drug, an administration time of the drug, and an administration amount of the drug. Also, the diameter of the lower portion 1930 may be adjusted based on a weight and a size of the upper portion 1910 and the middle portion 1920, a level of sustaining the solid drug, and an amount of time used for the lower portion 1930 to melt in the skin S.

In operation 2520, an initial middle portion in a shape of the cavity 1921 is formed on the lower portion 1930. Referring to FIG. (a) of 26, the method of manufacturing the microneedle containing the solid drug according to an example embodiment forms the initial middle portion in the shape of the cavity 1921 on the lower portion 1930 and a cavity top 1923 is open. Here, an area of the cavity 1921 may be provided in an upper area above the center of the middle portion 1920. However, depending on example embodiments, a position, a size, and a shape of the area of the cavity 1921 may be variously applied based on an administration point in time, an administration time, and an administration amount of the solid drug 2110. Also, the initial middle portion may represent a truncated pyramidal shape or a truncated conical shape that includes a form of the cavity 1921.

Depending on example embodiments, in operation 2520, the method of manufacturing the microneedle containing the solid drug according to an example embodiment may be represented as illustrated in (a) of FIG. 26 as a method of manufacturing the initial middle portion in the shape of the cavity 1921 using the 3D printing scheme and stacking the manufactured initial middle portion on the lower portion 1930 or manufacturing the initial middle portion in the truncated pyramidal shape or the truncated conical shape including the form of the cavity 1921 on the lower portion 1930.

In operation 2530, the solid drug 2110 that penetrates into the skin and melts is injected into the cavity 1921. Referring to (b) of FIG. 26, the method of manufacturing the microneedle containing the solid drug according to an example embodiment may provide the solid drug 2110 in the cavity 1921 through a movement method. Here, when the solid drug 2110 is injected into the cavity 1921, the solid drug 2110 may be absorbed into a material of the middle portion 1920. Therefore, a cavity surface may be coated with a waterproof material.

Here, the solid drug 2110 may be in a structure form in which a drug containing a drug is contained and a size and a type and a number of drugs contained in a structure in which a drug is contained may be adjusted based on a degree of a drug that penetrates into the skin S, performance of, a state of a subject to which the drug applies, and a melting time.

In operation 2540, the middle portion 1920 is formed by blocking the cavity top 1923 into which the solid drug 2110 is injected. Referring to (c) of FIG. 26, when the solid drug 2110 is injected into the cavity 1921, the method of manufacturing the microneedle containing the solid drug according to an example embodiment seals the cavity 1921 containing the solid drug 2110 in the middle portion 1920 by blocking the open cavity top 1923. Here, the method of manufacturing the microneedle containing the solid drug according to an example embodiment may block the cavity top 1923 with a material of the middle portion 1920 through a 3D printing scheme.

In operation 2550, the upper portion 1910 is formed on the middle portion 1920. Referring to (d) FIG. 26, the method of manufacturing the microneedle containing the solid drug according to an example embodiment may form the upper portion 1910 that is provided at a top end of the middle portion 1920 and facilitates penetration into the skin S. The upper portion 1910 may have a sharp tip shape based on a penetration direction for penetrating into the skin S. For example, the upper portion 1910 may be formed in a pyramidal shape or a conical shape, thereby facilitating penetration into the skin S.

Each of the upper portion 1910, the middle portion 1920, and the lower portion 1930 of the microneedle 1900 containing the solid drug according to an example embodiment may be formed of a different material. Although the upper portion 1910, the middle portion 1920, and the lower portion 1930 may be formed of the same water-soluble substance, the upper portion 1910 configured to facilitate penetration, the middle portion 1920 containing the solid drug 2110, and the lower portion 1930 configured to support the middle portion 1920 and to facilitate separation from the base portion 10 may be formed of different types of materials among water-soluble substances. For example, the upper portion 1910 and the lower portion 1930 may be formed of a material that melts within a short period of time compared to that of the middle portion 1920 such that a quantitative drug provided to the middle portion 1920 may be injected.

While the example embodiments are described with reference to specific example embodiments and drawings, it will be apparent to one of ordinary skill in the art that various alterations and modifications in form and details may be made in these example embodiments without departing from the spirit and scope of the claims and their equivalents. For example, suitable results may be achieved if the described techniques are performed in different order, and/or if components in a described system, architecture, device, or circuit are combined in a different manner, or replaced or supplemented by other components or their equivalents.

Therefore, other implementations, other example embodiments, and equivalents of the claims are to be construed as being included in the claims.

What is claimed is:

1. A microneedle in a structure of three or more layers, the microneedle comprising:
a middle portion configured to penetrate into the skin and formed of a compound containing a drug component;
a lower portion configured to support the middle portion and comprising an inner pillar shell having a hollow central portion with a preset radius; and
an upper portion provided at a top end of the middle portion and configured to facilitate penetration.

2. The microneedle of claim 1, wherein the lower portion comprises a single inner pillar shell or a plurality of inner pillar shells each formed with a preset radius and at a height of the lower portion and having the hollow central portion.

3. The microneedle of claim 2, wherein the inner pillar shell represents a core portion in a circular shape, an oval shape, a triangular shape, a quadrangular shape, or a polygonal shape.

4. The microneedle of claim 2, wherein the lower portion has a donut shape or a porous shape based on a size of the inner pillar shell and a number of inner pillar shells.

5. The microneedle of claim 1, wherein the upper portion and the middle portion have a pyramidal shape or a conical shape, and the lower portion has a pyramidal shape or a cylindrical shape.

6. A microneedle in a structure of three or more layers comprising a three-dimensional (3D) structure shell, the microneedle comprising:
a middle portion configured to penetrate into the skin and formed of a compound containing a drug component;
a lower portion configured to support the middle portion and comprising a 3D structure shell in which a plurality of units comprising a plurality of straight members extending in different directions are coupled; and
an upper portion provided at a top end of the middle portion and configured to facilitate penetration.

7. The microneedle of claim 6, wherein the lower portion represents the 3D structure shell that is a truss structure form in which a unit in which the plurality of straight members extending in different directions are arranged in a triangular shape is coupled and the plurality of units connected in the triangular shape are stacked.

8. The microneedle of claim 7, wherein the lower portion is configured to maintain a space between the plurality of straight members coupled as the unit and in the 3D structure shell and to adjust a melting rate after penetration into the skin by adjusting the space.

9. The microneedle of claim 6, wherein the upper portion and the middle portion have a pyramidal shape or a conical shape.

10. The microneedle of claim 9, wherein a bottom diameter of the middle portion is greater than a bottom diameter of the upper portion or a bottom diameter of the lower portion, and the bottom diameter of the upper portion is greater than the bottom diameter of the lower portion.

11. A microneedle containing a solid drug in a structure of three or more layers, the microneedle comprising:
a middle portion configured to penetrate into the skin and containing a solid drug in a cavity;
a lower portion configured to support the middle portion; and
an upper portion provided at a top end of the middle portion and configured to facilitate penetration.

12. The microneedle of claim 11, wherein the middle portion comprises a cavity in a groove form with a predetermined size therein and comprises, in the cavity, the solid drug in a structure in which a drug containing a drug is contained.

13. The microneedle of claim 12, wherein the middle portion is configured to seal the solid drug by blocking a top of the cavity that comprises the solid drug.

14. The microneedle of claim 13, wherein the middle portion comprises a plurality of solid drugs comprising different drugs.

15. The microneedle of claim 12, wherein a cavity surface in contact with the solid drug is coated with a waterproof material that does not react to the solid drug.
